(19) United States
(12) Patent Application Publication
(10) Pub. No.: US 2007/0021485 A1
(43) Pub. Date: Jan. 25, 2007

(54) Aryl (or heteroaryl) azolylcarbinols

(76) Inventors: Antonio Farre Gomis, Barcelona (ES); Blas Andaluz-Mataro, Barcelona (ES); Ramon Merce Vidal, Barcelona (ES); Jordi Frigola-Constansa, Barcelona (ES)

Correspondence Address: COOPER & DUNHAM, LLP, 1185 AVENUE OF THE AMERICAS, NEW YORK, NY, 10036, US

(21) Appl. No.: 11/187,697

(22) Filed: Jul. 22, 2005

(51) Int. Cl. A61K 31/415; C07D 231/02

(52) U.S. Cl. 514/406; 548/375.1

(57) ABSTRACT

The present application provides a method of treating a subject suffering from a form of urinary incontinence which comprises administering to the subject an amount of a compound having the structure: 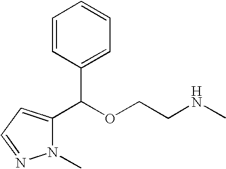 effective to treat the subject, wherein the compound is administered in a suitable form.

### FIELD OF THE APPLICATION

This application claims benefit of U.S. Ser. No. 10/902,262, filed Jul. 30, 2004, the contents of which are hereby incorporated herein by reference.

Within this application certain publications and patent documents are cited to define the state of the art. The disclosures of these publications and documents in their entireties are hereby incorporated by reference into this application.

The present application provides the use of derivatives of aryl (or heteroaryl) azolylcarbinols of general formulas (I), (II), (III) or (IV) and their physiologically acceptable salts, as medicinal products for human and/or animal therapeutics for the treatment of various diseases including urinary incontinence and pain.

### STATE OF THE ART

Depression is a common problem that affects a large segment of the population in all age groups. Antidepressants account for almost half of worldwide, psychiatric related drug sales.

In this regard, substance P, a takchykinin, is present in the brain at relatively high concentrations. It has been suggested that substance P may function as a neurotransmitter. Some nerve terminals containing

Substance P have synaptic contacts with cholinergic neurons, and the stimulation of the NK1 receptors by substance P results in an increase of the release of acetylcholine (J. J. Anderson, J. Pharmacol. Exp. Ther., 1995, 274, 928-936).

Substance P has been implicated in the pathophysiology of several neuropsychiatric disorders, including schizophrenia, drug addiction, cognitive disorders, manic depressive psychosis, locomotive disorders, sexual dysfunction, and depression. A clear relation between depressive states and levels of substance P may be assumed, since products which act as inhibitors of substance P have a clear anti-depressive component when studied in several laboratory animal models.

The major neurochemical abnormality in depression is thought to be a deficiency in monoamine neurotransmitter function, especially serotonin and noradrenaline function, and possibly dopamine. Given the high incidence of the different forms of depression and related disorders, there exist several pharmacological ways to undertake the problem. Initial treatments for depression focused on attempts to inhibit the metabolic breakdown of serotonin, noradrenaline and adrenaline using inhibitors of the enzyme monoamine oxidase.

Another pharmacological approach has been the use of tricyclic compounds which show mixed inhibitory activity on noradrenaline and serotonin reuptake, although most show undesired secondary effects. More recently, a new group of compounds, the selective serotonin reuptake inhibitors were introduced. They present a clear improved side effect profile as compared to other pharmacological approaches.

In the course of the studies of the metabolic pathways of cizolirtine, compounds were isolated with a significant binding affinity for the serotonin reuptake transporter. Although the activity of cizolirtine as antidepressant is very low in laboratory animal models, the compounds described herein behave as an antidepressant in these tests with an efficacy similar to that of the described selective serotonin reuptake inhibitors.

More particularly, it has been discovered that new derivatives of aryl or heteroaryl azolylcarbinoles as well as their salts, are useful in the manufacture of medicaments, useful in human therapy, for the treatment of certain disorders of the central nervous system, especially disorders mediated by excess of substance P or depression but also for other indications, particularly urinary incontinence.

### SUMMARY OF THE APPLICATION

The present application provides a method of treating a subject suffering from a form of urinary incontinence which comprises administering to the subject an amount of a compound having the structure: 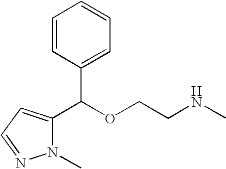
effective to treat the subject, wherein the compound is administered in a suitable form.

Useful forms of the compound include the racemate, pure stereoisomers, especially enantiomers or diastereomers or mixtures of stereoisomers, enantiomers or diastereomers, in any suitable ratio. The compound may be used in the form shown or in form of an acid, a base, or a salt, especially a physiologically acceptable salt; or in form of a solvate.

### BRIEF DESCRIPTION OF THE FIGURES

FIG. 1 is a diagrammatic representation of the putative mechanism of action of the N-desmethyl metabolite of cizolirtine showing how the metabolite enters the mPFC and PAG areas of the brain increasing serotonin and noradrenaline uptake and thereby decreasing substance P and cGRP resulting in a decrease in the symptoms of stress urinary incontinence and urge incontinence.

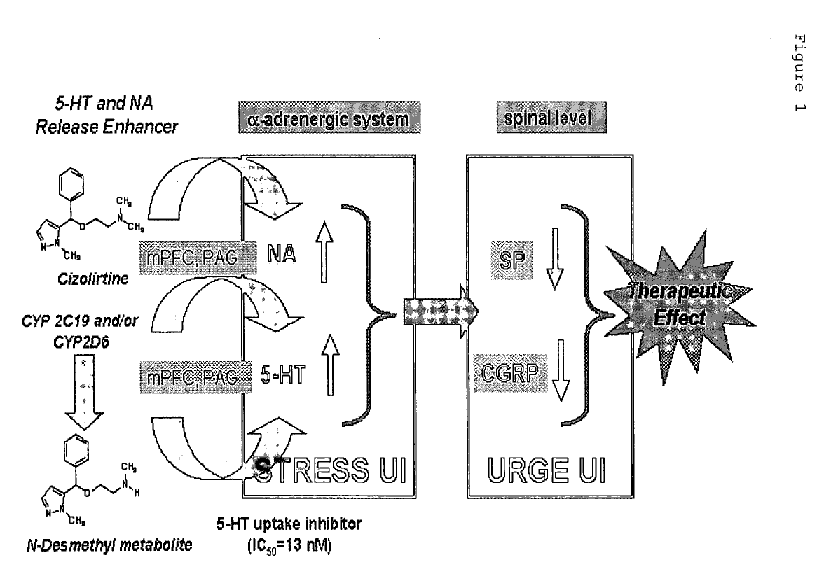

FIG. 2 shows a comparison of the percentage of change of rat bladder contraction amplitude in anesthetized rat bladders treated with (R)-(+)-2-((1-methyl-1H-pyrazol-5-yl)(phenyl)methoxy-N-methylethanamine citrate, (S)-(−) -2-((1-methyl-1H-pyrazol-5-yl)(phenyl)methoxy-N-methylethanamine citrate, and the control vehicle oxybutinyn.

FIG. 3 shows a comparison of urodynamic parameters of the isovoumetric rhythmic bladder contractions in the anesthetized rat treated treated with_{—}2-((1-methyl-1H-pyrazol-5-yl)(phenyl)methoxy-N-methylethanamine, cizolirtine, and duloxetine to those treated with a control vehicle.

FIG. 4 shows the relative effect of treatment of anesthetized rat bladders with 2-((1-methyl-1H-pyrazol -5-yl) (phenyl)methoxy-N-methylethanamine, cizolirtine, and duloxetine on acetic acid-induced bladder hyperactivity compared to the control vehicle. Specifically measured is the number of micturitions and volume for first micturition.

FIG. 5 shows the effect of anesthetized rat bladders with 2-((1-methyl-1H-pyrazol-5-yl)(phenyl)methoxy-N-methylethanamine, cizolirtine, and duloxetine, on acetic acid-induced bladder hyperactivity compared to the control vehicle. Specifically measured was the effect on micturition volume, intravesical pressure increase, and AUC of intravesical pressure of control vehicle and treated anesthetized rats.

### DETAILED DESCRIPTION OF THE INVENTION

This application provides a method of treating a subject suffering from a form of urinary incontinence which comprises administering to the subject an amount of a compound having the structure: 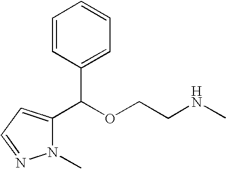
effective to treat the subject, wherein the compound is administered in a suitable form.

Surprisingly, it is found that a compound as described above is active in urinary incontinence. Therefore, an aspect of the application is the use of such a compound in the preparation of a medicament for the treatment of forms of urinary incontinence, including urge incontinence, hyperreflexia; urinary stress incontinence, mixed incontinence and enuresis.

Urinary incontinence, is a urinary disorder which can be defined as the involuntarily discharge of urine. This effect can be demonstrated objectively. This functional disorder of the bladder is a health problem of increasing social and hygienic relevance for those that suffer from it. Urinary incontinence is estimated to occur in approximately 1.5 to 5% of men, and 10 to 30% of women, between 15 and 64 years old. Moreover, in the non-hospitalized population sector over 60 years old, the prevalence ranges from 15% to 35%. When hospitalized patients over 60 years old are considered, the incidence is even higher.

Urinary incontinence can be considered as a symptom or as a pathological condition. The following are possible classifications of this functional disorder.

Imperative micturition or urge incontinence. This form of urinary incontinence occurs when the involuntary discharge of urine is accompanied by an intense desire to urinate (urgency). This can be separated into motor urgency incontinence or sensitive urgency incontinence. Motor urgency incontinence is associated with hyperactivity of the detrusor muscle and/or reduced distensibility of the detrusor. Hyperactivity is characterised by involuntary contractions of the detrusor during the filling stage, either spontaneous or provoked, that the patient cannot totally suppress. Hyperactivity of the detrusor muscle can occur when there is obstruction of the exiting urinary flow, inflammation and conditions in which the bladder is irritated, or it can be of unknown etiology (idiopathic).

Hyperreflexia, is described as a condition that presents uncontrolled contractions of the detrusor muscle associated with neurological disorders such as multiple sclerosis or plaque sclerosis, sequelae of medular traumatisms, or Parkinson's disease.

Urinary stress incontinence is typically due to a defective urethral closure mechanism. There is involuntary discharge of urine in the absence of detrusor contraction that occurs when the intravesical pressure exceeds the pressure in the urethra. Involuntary discharge occurs when some physical exertion is made such as jumping, coughing, going down stairs etc. One additional factor can be due to structural changes in the urethra due to menopausal hypooestrogenia.

Mixed incontinence refers to the existence of both urgency incontinence and stress incontinence.

Enuresis refers to any involuntary loss of urine and more specifically to incontinence during sleep. It most often applies to children with a higher incidence in boys, particularly those up to 5 years of age.

For additional information concerning these terms, Abrams et al, Neurology and Urodynamics 21:167-178 (2002), the content of which is hereby incorporated by reference, may be considered.

This application provides a method of treating a subject suffering from a form of urinary incontinence which comprises administering to the subject an amount of a compound having the structure: 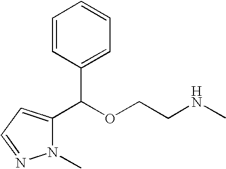
effective to treat the subject, wherein the compound is administered in a suitable form.

In an embodiment, the compound is in the form of a racemic mixture.

In a further embodiment, the compound is in the form of a pure stereoisomer or as a mixture of stereoisomers in a suitable relative ratio.

In another embodiment, the compound is in the form of an enantiomer, a diastereomer, or a mixture of enantiomers and/or diasteromers in a suitable relative ratio.

In yet another embodiment, the compound is in the form of an acid, a base, a phisiolocially acceptable salt, or a solvate. In an embodiment, the compound is in the form of a solvate, and the solvate is a hydrate.

In an embodiment, the compound is in the form of an enantiomer selected from the group consisting of:
(R)-(+)-2-((1-methyl-1H-pyrazol-5-yl)(phenyl)methoxy-N-methylethanamine
(S)-(−)-2-((1-methyl-1H-pyrazol-5-yl)(phenyl)methoxy-N-methylethanamine
(R)-(+)-2-((1-methyl-1H-pyrazol-5-yl)(phenyl)methoxy-N-methylethanamine citrate
(S)-(−)-2-((1-methyl-1H-pyrazol-5-yl)(phenyl)methoxy-N-methylethanamine citrate

In another embodiment, the compound is in the form of a pharmaceutically acceptable salt which is a citrate.

In an embodiment, the subject is a human being.

In a further embodiment, the effective amount is between 0.167 and 13.333 mg/kg body weight of the subject/day.

In another embodiment, the effective amount is between 0.167 and 3.333 mg/kg body weight of the subject/day

In yet another embodiment, the effective amount is between 0.333 and 1.667 mg/kg body weight of the subject/day.

In an embodiment, the effective amount is between 10 and 800 mg administered daily. Preferably, between 10 and 200 mg administered daily; more preferably between 20 and 100 mg administered daily;

In another embodiment, the effective amount is 200 mg administered daily; preferably 100 mg administered daily; more preferably 50 mg administered daily; or 20 mg administered daily.

In yet another embodiment, the compound is administered twice per day.

In an embodiment, the compound is present in a formulation that contains a coating agent and the formulation is administered daily. In a another embodiment, the coating agent is a controlled release coating agent.

In another embodiment, the formulation comprises any of the following: sodium croscarmelose; colloidal silica dioxide; a salt with stearic acid; providone; microcrystalline cellulose; lactose monohydrate; or polyethylene glycol.

In yet another embodiment, the compound being is administered in the form of a tablet or capsule.

In an embodiment the compound is administered in the form of an immediate release formulation.

In another embodiment, the subject is a woman. In an embodiment, the woman is an elderly woman.

In yet another embodiment, the subject is a man. In an embodiment the subject is an elderly man.

In another embodiment, the subject is a child.

In an embodiment, the form of urinary incontinence is urge urinary incontinence; stress urinary incontinence or urinary stress incontinence; hyperreflexive urinary incontinence; or enuresis.

This application also provides a use of a compound having the structure: 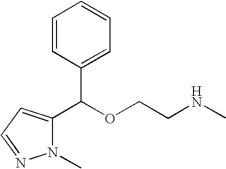
in a suitable form for the preparation of a medicament comprising an effective amount for treatment of a form of urinary incontinence

In an embodiment, compound is in the form of a racemic mixture.

In another embodiment, the compound is in the form of a pure stereoisomer or as a mixture of stereoisomers in a suitable relative ratio.

In yet another embodiment, the compound is in the form of an enantiomer, a diastereomer, or a mixture of enantiomers and/or diasteromers in a suitable relative ratio.

In an embodiment, the compound is in the form of an acid, a base, a physiologically acceptable salt, or a solvate.

In an embodiment, the compound is in the form of a solvae, and the solvate is a hydrate.

In another embodiment, the compound is in the form of an enantimer selected from the the group consisting of:
(R)-(+)-2-((1-methyl-1H-pyrazol-5-yl)(phenyl)methoxy-N-methylethanamine
(S)-(−)-2-((1-methyl-1H-pyrazol-5-yl)(phenyl)methoxy-N-methylethanamine
(R)-(+)-2-((1-methyl-1H-pyrazol-5-yl)(phenyl)methoxy-N-methylethanamine citrate
(S)-(−)-2-((1-methyl-1H-pyrazol-5-yl)(phenyl)methoxy-N-methylethanamine citrate

In yet another embodiment, the comound is in the form of a pharmaceutically acceptable salt which is a citrate.

In an embodiment, the effective amount is between 0.167 and 13.333 mg/kg body weight of the subject/day; preferably, between 0.167 and 3.333 mg/kg body weight of the subject/day; and more preferably, between 0.333 and 1.667 mg/kg body weight of the subject/day.

In another embodiment, the effective amount is between 10 and 800 mg; preferably, 10 and 200 mg; and more preferably, between 20 and 200 mg;

In yet another embodiment, effective amount is 200 mg; preferably, 100 mg; more preferably, 50 mg; or 20 mg.

In an embodiment, the compound is present in a formulation comprising any of the following: sodium croscarmelose; colloidal silica dioxide; a salt with stearic acid; providone; microcrystalline cellulose; lactose monohydrate; or polyethylene glycol.

In another embodiment, the compound is in the form of a tablet or capsule.

In yet another embodiment, the compound is in the form of an immediate release formulation.

In an embodiment, the form of urinary incontinence is urge urinary incontinence; stress urinary incontinence or urinary stress incontinence; hyperreflexive urinary incontinence; or enuresis.

In another embodiment, the compound is present in a formulation that contains a coating agent. In an embodiment, the coating agent is a controlled release coating agent.

An additional object of this application is to provide an aryl or heteroaryl azolylcarbinole derivative of general formula (I), (II), (III) or (IV) 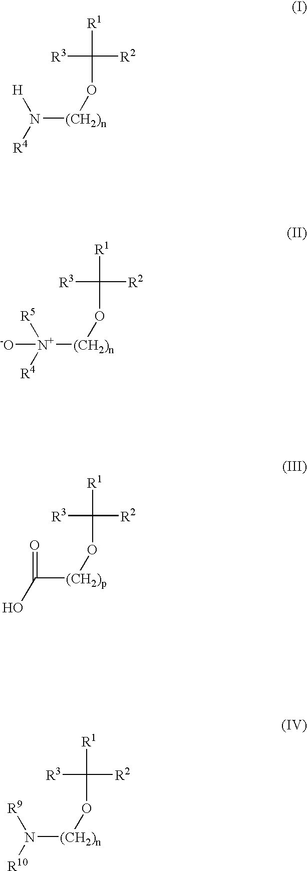
in which
R¹ represents a hydrogen atom or a lower alkyl group from C₁ to C₄;
R² represents a phenyl radical or a thienyl radical, with no substitutions or optionally with 1, 2 or 3 equal or different substituents, selected from the group comprised of hydroxy, fluoride, chloride, bromide, methyl, trifluoromethyl and methoxy, or —O-(glucoronic acid);
R³ represents a five-armed nitrogenated aromatic heterocycle that contains one to three nitrogen atoms, without substitutions or optionally substituted by 1 or 2 equal or different substituents selected from a group comprised by fluoride, chloride, bromide and methyl;
R⁴ represents H or C₁₋₄-Alkyl;
R⁵ represents H or C₁₋₄-Alkyl;
R⁹ is glucoronic acid;
R¹⁰ represents C₁₋₄-Alkyl; and
n is 1 to 4 or p is 1 to 3
optionally in the form of its racemate, pure stereoisomers, especially enantiomers or diastereomers or in the form of mixtures of stereoisomers, especially enantiomers or diastereomers, in any suitable ratio; in the form shown or in form of the acid or base or in form of a salt, especially a physiologically acceptable salt, or in form of a solvate, especially a hydrate.

Excluded from this are:
2-((1-methyl-1H-pyrazol-5-yl)(phenyl)methoxy)ethanamine,
2-((1-methyl-1H-pyrazol-5-yl)(phenyl)methoxy)-N-methylethanamine,
N-oxo-2-((1-methyl-1H-pyrazol-5-yl)(phenyl)methoxy)-N,N-dimethylethanamine;
2-((1-methyl-1H-pyrazol-5-yl)(phenyl)methoxy)acetic acid;
2-((1-methyl-1H-pyrazol-5-yl)(thiophen-2-yl)methoxy)-N-methylethanamine.

The compounds falling within formulas (I), (II), (III) and (IV) may also be included within a more general formula (VIII): 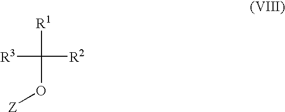
wherein R¹, R² and R³ have the meanings already mentioned and Z represents one of the following groups: 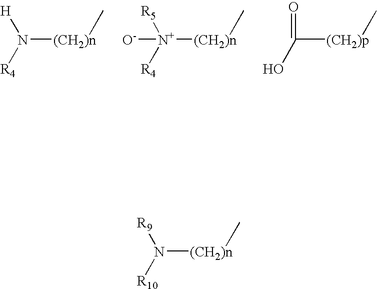
where
R⁴ represents H or C₁₋₄-Alkyl;
R⁵ represents H or C₁₋₄-Alkyl;
R⁹ is glucoronic acid;
R¹⁰ represents C₁₋₄-Alkyl; and
n is 1 to 4 or p is 1 to 3
optionally in the form of its racemate, pure stereoisomers, especially enantiomers or diastereomers or in the form of mixtures of stereoisomers, especially enantiomers or diastereomers, in any suitable ratio; in the form shown or in form of the acid or base or in form of a salt, especially a physiologically acceptable salt, or in form of a solvate, especially a hydrate.

These compounds are mentioned as metabolites (and only as metabolites) of cizolirtine in L. Martinez et al., Absorption, distribution, metabolism and excretion of cizolirtine, a new analgesic compound, in rat and dog, Xenobiotica, 1999 (29) 8, 859-871 or as metabolites (and again only as metabolites) in Puig S., et al., “Validation of a chromatographic method to determine E-6006 and its metabolite E-6332 in rat and dog plasma by solid-phase extraction and capillary gas chromatography. Application in pharmacokinetics,” J. Pharm. Biomed. Anal. 24 (2001) 887-896.

The new derivatives of aryl or heteroaryl azolylcarbinoles described herein are surprisingly useful in the treatment of disorders related to substance P, especially in the treatment of depression but also in other indications.

These new compounds are all related to compounds described in EP 0 289 380 or U.S. Pat. No. 5,017,596 (describing i.a. cizolirtine) as well as EP 1 072 266 or U.S. Pat. No. 6,410,582, which patents and applications are all hereby incorporated herein by reference.

In the context of this application, alkyl radicals are understood as meaning saturated and unsaturated, branched or unbranched hydrocarbons, which can be unsubstituted or mono- or polysubstituted. In these radicals, C1-2-alkyl represents C1- or C2-alkyl, C1-3-alkyl represents C1-, C2- or C3-alkyl, C1-4-alkyl represents C1-, C2-, C3- or C4-alkyl, C1-5-alkyl represents C1-, C2-, C3-, C4-, or C5-alkyl, C1-6-alkyl represents C1-, C2-, C3-, C4-, C5- or C6-alkyl, C1-7-alkyl represents C1-, C2-, C3-, C4-, C5-, C6- or C7-alkyl, C1-8-alkyl represents C1-, C2-, C3- , C4-, C5-, C6-, C7- or C8-alkyl, C1-10-alkyl represents C1-, C2-, C3-, C4-, C5-, C6-, C7-, C8-, C9- or C10-alkyl and C1-18-alkyl represents C1-, C2-, C3-, C4-, C5-, C6-, C7-, C8-, C9-, C10-, C11-, C12-, C13-, C14-, C15-, C16-, C17- or C18-alkyl. The alkyl radicals are preferably methyl, ethyl, vinyl (ethenyl), propyl, allyl (2-propenyl), 1-propinyl, methylethyl, butyl, 1-methylpropyl, 2-methylpropyl, 1,1-dimethylethyl, pentyl, 1,1-dimethylpropyl, 1,2-dimethylpropyl, 2,2-dimethylpropyl, hexyl, 1-methylpentyl, CHF₂, CF₃ or CH₂OH.

In connection with alkyl—unless expressly defined otherwise—the term substituted in the context of this application is understood as meaning replacement of at least one hydrogen radical by F, Cl, Br, I, NH₂, SH or OH, “polysubstituted” radicals being understood as meaning that the replacement takes effect both on different and on the same atoms several times with the same or different substituents, for example three times on the same C atom, as in the case of CF₃, or at different places, as in the case of —CH(OH)—CH═CH—CHCl₂. Particularly preferred substituents here are F, Cl and OH. In respect of cycloalkyl, the hydrogen radical can also be replaced by O—C1-3-alkyl or C1-3-alkyl (in each case mono- or polysubstituted or unsubstituted), in particular methyl, ethyl, n-propyl, i-propyl, CF₃, methoxy or ethoxy.

The term (CH₂)3-6 is to be understood as meaning —CH₂—CH₂—CH₂—, —CH₂—CH₂—CH₂—CH₂, —CH₂—CH₂—CH₂—CH₂—CH₂— and —CH₂—CH₂—CH₂—CH₂—CH₂—CH₂—, (CH₂)1-4 is to be understood as meaning —CH₂—, —CH₂—CH₂—, —CH₂—CH₂—CH₂-and —CH₂—CH₂—CH₂—CH₂—, (CH₂)4-5 is to be understood as meaning —CH₂—CH₂—CH₂—CH₂— and —CH₂—CH₂—CH₂—CH₂—CH₂—, etc.

An aryl heterocycle (heteroaryl) is understood as meaning a heterocyclic ring system which have at least one unsaturated ring and can contain one or more heteroatoms from the group consisting of nitrogen, oxygen and/or sulfur and can also be mono- or polysubstituted. Examples which may be mentioned from the group of heteroaryls are furan, benzofuran, thiophene, benzothiophene, pyrrole, pyridine, pyrimidine, pyrazine, quinoline, isoquinoline, phthalazine, benzo-1,2,5-thiadiazole, benzothiazole, indole, benzotriazole, benzodioxolane, benzodioxane, carbazole and quinazoline.

Here, in connection with heteroaryl, substituted is understood as meaning substitution of the aryl or heteroaryl by R, OR, a halogen, preferably F and/or Cl, a CF₃, a CN, an NO₂, an NRR, a C1-6-alkyl (saturated), a C1-6-alkoxy, a C3-8-cycloalkoxy, a C3-8-cycloalkyl or a C2-6-alkylene.

The term glucoronic acid means a radical of this structure:

In the context of this application, the term “coating agent” is to be understood to be a chemical substance added to a coating or as a coating to produce an effect on the surface of the pharmaceutical composition or one of its subunits. The effects desired include release control, chemical stability enhancement (e.g. gastrointestinal coating, light exclusion), physical stability enhancement (hardening, confining/conserving the surface), or e.g. coloring. In a preferred sense, “coating agent” is understood as “release control coating agent,” which is used herein synonymously with “controlled release coating agent,” a chemical substance added to or as a coating to control the release of the active compound/principle from the pharmaceutical composition or from one of its subunits. A preferred coating agent for this application is ethylcellulose. In a preferred embodiment, the ethylcellulose is used as a pseudo-latex containing plasticizer already incorporated into dispersed particles in water or “in situ” with ethylcellulose plus organic solvents or solvent mixture. Other coating agents, especially for release control, commonly used and also suitable for the application are (as examples and not limiting):
Fats and waxes (e.g., glyeril monostearate, beeswax, carnauba wax . . . ). These coatings normally function by erosion and not by diffusion as ethylcellulose.
Acrylic polymers (polymethacrilates): Eudragit series
Aquacoat (FMC): Ethylcellulose dispersion, a pseudo-latex stabilized in water with sodium lauryl sulphate
Other types of cellulose: HPMC (Hydroxypropilmethylcellulose), HPC (Hydroxypropilcellulse), Methylcellulose, etc.

The term “solvate” is understood in particular, in the context of this application as a compound formed by salvation (the combination of solvent molecules with molecules or ions of the solute).

The term “salt” means any form of the active substance according to the application, in which this assumes an ionic form and is loaded/charged, and coupled with a counterion (a cation or anion) found in solution.

This also indicates complexes of the active substance with other molecules and ions, particularly complexes formed via ionic interactions. In particular, this indicates (and this is also a preferred embodiment of this application) physiologically acceptable salts, particularly physiologically acceptable salts with anions or acids, or also salts made from a physiologically acceptable acid or a physiologically cation.

As defined by this application, the term “physiologically acceptable salt with anions or acids” means salts of at least one of the combinations according to the application—mostly, e.g. those protonated at the nitrogen atom,—as a cation with at least one anion, which are physiologically acceptable—in particular while using in humans and/or mammals. In particular this indicates, as defined by this application, the salt made from a physiologically acceptable acid, namely salts of the respective active substances with inorganic or organic acids, which are physiologically acceptable—in particular while using in humans and/or mammals. Examples for physiologically acceptable salts of certain acids are the salts of: hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid, citric acid, glutamic acid, 1,1 dioxo-1.2-dihydrolb⁶-benzo[d]isothiazol-3-one (saccharic acid), monomethyl sebacic acid, 5-oxo-proline, hexan-1-sulfonic acid, nicotinic acid, 2-, 3- or 4-aminobenzoic acid, 2,4,6-trimethylbenzoic acid, α-lipoic acid, acetylglycine, acetylsalicylic acid, hippuric acid, and/or aspartic acid. Especially preferred is the hydrochloride salt. Included are also salts of alkali metals and alkaline earth metals and with NH₄

As defined herein, a “salt made from a physiologically acceptable acid” means the salts of the respective active substance with inorganic or organic acids, which are physiologically acceptable—in particular while using in humans and/or mammals. Especially preferred is the hydrochloride. Examples of physiologically acceptable acids are: hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid, citric acid, glutamic acid, 1,1-dioxo-1.2-dihydrolb⁶-benzo[d]isothiazol-3-one (saccharic acid), monomethyl sebacic acid, 5-oxo-proline, hexan-1-sulfonic acid, nicotinic acid, 2-, 3-, or 4-aminobenzoic acid, 2,4,6-trimethylbenzoic acid, α-Lipon acid, acetylglycine, acetylsalicylic acid, hippuric acid, and/or aspartic acid.

As defined herein, the term “physiologically acceptable salts with cations or bases” means salts having at least one of the combinations according to the application—mostly a (deprotonated) acid—as an anion with at least one, preferably inorganic, cation, which are physiologically acceptable—in particular while using in humans and/or mammals. However, especially preferred are the salts of the alkali metals and alkaline earth metals, but also those with NH₄⁺, but particularly (mono) or (di) sodium, (mono) or (di) potassium, magnesium, or calcium salts.

As defined herein the term “salts made from a physiologically acceptable cation” means salts of at least one of the respective combinations of an anion with at least one inorganic cation, which are physiologically acceptable—in particular while using in humans and/or mammals. However, especially preferred are the salts of the alkali metals and alkaline earth metals, but also those with NH₄⁺, but particularly (mono) or (di) sodium, (mono) or (di) potassium, magnesium, or calcium salts.

The citrate is particularly preferred.

Given its favorable pharacodynamic properties, the compound described above may be used in human and animal subjects to cure, or at least relieve urinary incontinence.

In humans, the dose of the compounds to be administered depends on the severity of the condition to be treated. It is typically between 10 and 800 mg/day; preferably between 10 and 200 mg/day; and most preferably between 20 and 100 mg/day. The compound may be administered in many standard dosage forms known in the art, for example, in the form of a capsule or a tablet.

Formulations, i.e. pharmaceutical compositions, containing the compound as active ingredient may optionally include at least one auxiliary material and/or additive.

The following examples are provided to illustrate the application and are not intended to limit its scope.

### EXAMPLE 1

### A. Animal Studies

(1) Isovolumetric Rhythmic Bladder Contractions in the Anesthetized Rat

(a) Amplitude and Rate of the Contractions

(i) Method

Rat urinary bladder catheterized:

A pressure transducer is used to measure intravesical pressure (recording rate and amplitude of bladder contractions).

An infusion pump is used to fill bladder by 0.1 ml increments of saline until rhythmic contractions. After 15 minutes of constant interval bladder contractions, (R)-(+)-2-((1-methyl-1H-pyrazol-5-yl)(phenyl)methoxy-N-methylethanamine citrate in 6.83, 13.7, and 27.3 mg/kg dosages; (S)-(−)-2-((1-methyl-1H-pyrazol-5-yl)(phenyl)methoxy-N-methylethanamine citrate in 6.83, 13.7 and 27.3 mg/kg dosages; and oxybutinyn in a 1 mg/kg dose were administered by iv. Measurements were made before and then during 15 minute intervals after each administration.

(ii) Outcome

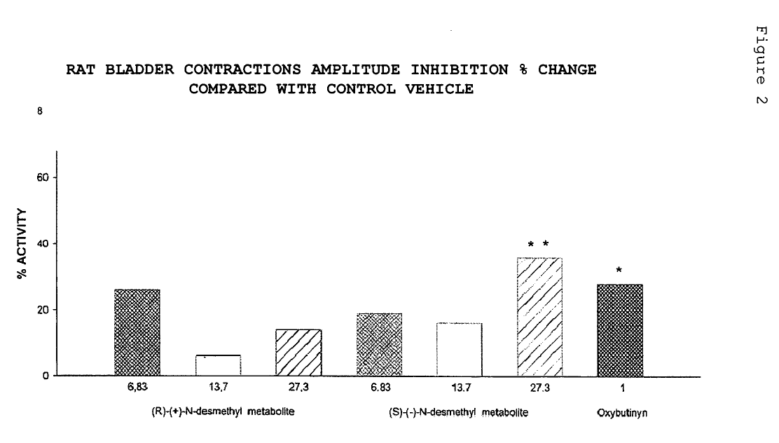

(R)-(+)-2-((1-methyl-1H-pyrazol-5-yl)(phenyl)methoxy-N-methylethanamine and (S)-(−)-2-((1-methyl-1H-pyrazol-5-yl)(phenyl)methoxy-N-methylethanamine showed a pattern of activity similar to oxybutynin. They inhibited the amplitude of the bladder contractions, without modifying the rate, showing that the N-desmethyl metabolite can contribute to the activity of cizolirtine. (See FIG. 2)

(b) Urodynamic Parameters

(i) Method

Rat urinary bladder was catheterized. A pressure transducer was used to measure intavesical pressure in anesthetized rats treated with 1 mg/kg duloxetine, 10 mg/kg cizolirtine, 10 mg/kg N-desmethyl metabolite or a control vehicle. Recordings were made of the infusion volume, pressure needed for micturition, number of micturitions during the infusion.

An infusion pump was used to fill bladder with saline until micturition. After stabilization, treatment compounds or control vehicle were administered by iv. Fifteen minutes after administration, saline infusion was started and urodynamic parameters were again analyzed. The number of micturitions, volume for first micturition, mean volume for micturition, and AUC of intravesical pressure were measured for each of the treatment groups and compared to the control.

(ii)Outcome

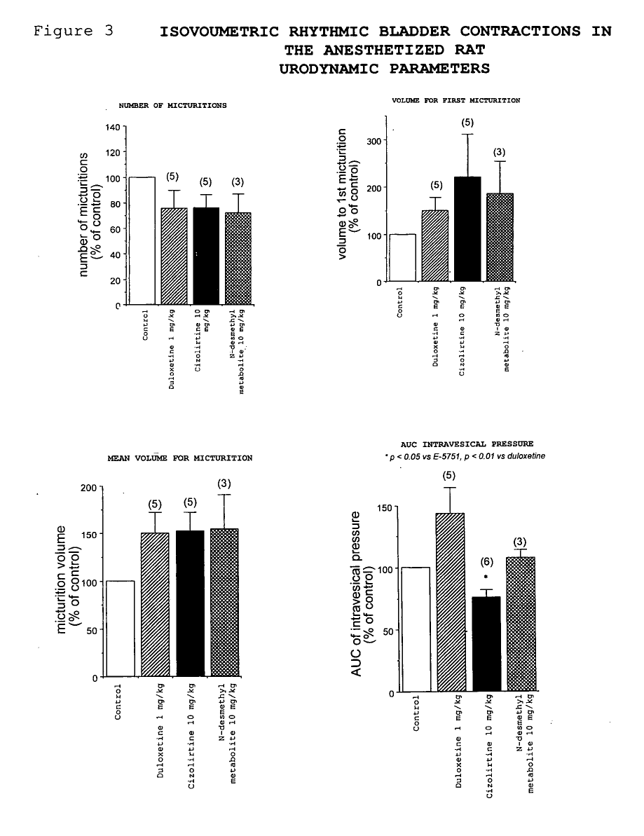

The N-desmethyl metabolite, like cizolirtine or duloxetine, decreased the number of micturitions and increased the volume for the first micturition and the mean volume of micturition. (See FIG. 3)

Again, this shows that N-desmethyl metabolite can contribute to the cizolirtine activity.

(2) Isovolumetric Rhythmic Bladder Contractions in the Anesthetized Rat

(a) Protection Against Acetic Acid-induced Hyperactivity: Urodynamic Parameters

Anesthetized rat bladders were treated with CH₃OCOOH, CH₃OCOOH and 1 mg/kg duloxetine, CH₃OCOOH and 5 mg/kg cizolirtine, CH₃OCOOH and 10 mg/kg cizolirtine, or CH₃OCOOH and 10 mg/kg N-desmethyl metabolite. The number of micturitions, the volume for first micturition, micturition volume, intravesical pressure increase, and AUC of intravesical pressure were measured for each of the treatment groups and the control.

(i) Outcome

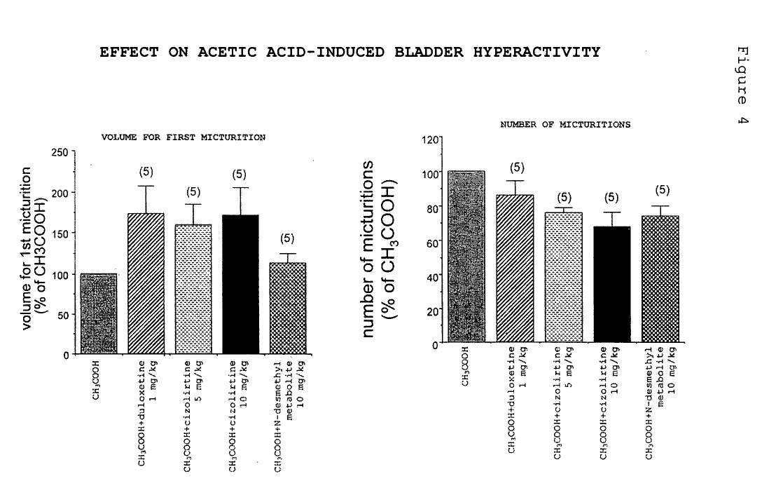

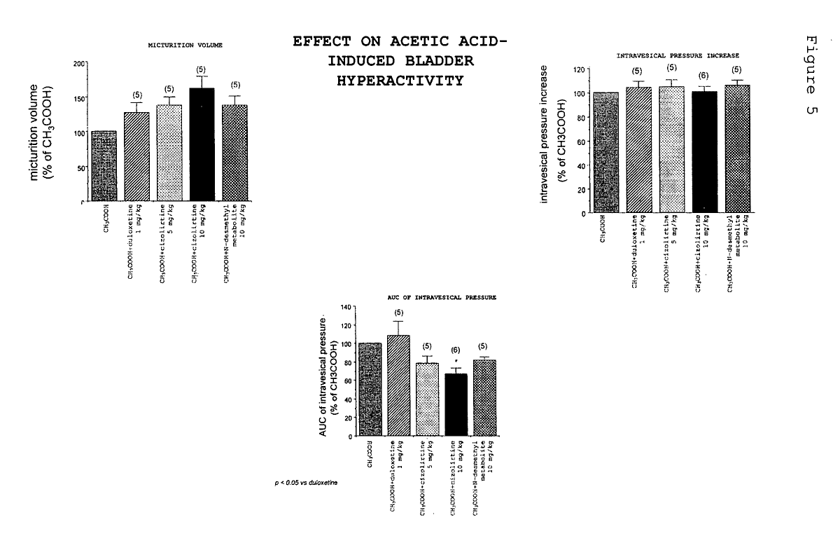

The N-desmethyl metabolite at 10 mg/kg, like cizolirtine at 5 and 10 mg/kg and duloxetrine at 1 mg/kg reduced the number of micturitions and increased the volume for the first micturition compared to the control (See FIG. 4). Similarly, it increased the number of micturitions (See FIG. 5).

Thus, N-desmethyl metabolite (10 mg/kg), like cizolirtine (5, 10 mg/kg, i.v.) and duloxetrine (1 mg/kg), protected the rat bladder against the acetic acid-induced hyperactivity.

Once again, this shows N-desmethyl metabolite can contribute to cisolirtine activity. (See FIGS. 4 and 5).

### EXAMPLE 2

### Clinical Study A

In a placebo controlled clinical trial 79 patients are randomized. Patients are from both genders with ages between 18 and 80 years (inclusive). They have urinary incontinence secondary to overactive bladder (detrusor hyperreflexia or instability) or idiopathic urge incontinence confirmed by the medical history and urodynamic study.

One group of patients is treated with 2-((1-methyl-1H-pyrazol-5-yl)(phenyl)methoxy-N-methylethanamine, 50 mg, twice a day, formulated as a tablet for oral administration. Another group is treated with placebo in matching tablets, twice a day, with administration by oral route. The patients are treated for 84 days.

Efficacy is measured by the difference from baseline in the mean number of leakages, micturitions, urgencies and voidings/24 hours as provided by a 7-day frequency-volume chart in the end of the study visit.

The primary efficacy analysis is based on the PP population. The treatment groups are compared with respect to the treatment effect, defined as the difference between treatment groups for changes from baseline in the number of voidings per 24 hours.

Fifty mg dose of 2-((1-methyl-1H-pyrazol-5-yl)(phenyl)methoxy-N-methylethanamine shows a significant reduction in the mean number of daily voidings, leakages, urgencies and micturitions compared with placebo.

Additionally, the percentage of responders is compared based on an analysis of the number of patients having <8 voidings/day or experiencing complete dryness or both, and statistical significance determined.

Such a clinical trial demonstrates the potential of 2-((1-methyl-1H-pyrazol-5-yl)(phenyl)methoxy-N-methylethanamine 50 mg twice a day by oral route for improving urge incontinence caused by overactive bladder, with clinically significant improvements compared to placebo in the majority of efficacy variables considered.

### EXAMPLE 3

### Clinical Study B

In a placebo controlled clinical trial 135 patients are randomized. Patients are from both genders with ages between 18 and 80 years (inclusive). They have urinary incontinence secondary to overactive bladder (detrusor hyperreflexia or instability) or idiopathic urge incontinence confirmed by the medical history and urodynamic study.

One group of patients is treated with 2-((1-methyl-1H-pyrazol-5-yl)(phenyl)methoxy-N-methylethanamine 100 mg formulated as an oral administration (twice daily). Another group is treated with placebo matching capsules, three per day (morning, afternoon and evening), with administration by oral route. The patients are treated for 84 days.

Efficacy is measured by the difference from baseline in the mean number of voidings/24 hours as provided by a 7-day frequency-volume chart in the end of study visit.

The primary efficacy analysis is based on the PP population. The treatment groups are compared with respect to the treatment effect, defined as the difference between treatment groups for changes from baseline in the number of voidings per 24 hours.

Additionally, the percentage of responders is compared based on analysis of the number of patients having <8 voidings per day or experienced complete dryness or both., and statistical significance determined.

The potential of 2-((1-methyl-1H-pyrazol-5-yl)(phenyl)methoxy-N-methylethanamine at 100 mg administered by oral route to improve bladder overactivity is demonstrated in such a clinical trial.

### EXAMPLE 4

Example of Formulation for Injectable (im/iv)

| | |
|---|---|
| 2-((1-methyl-1H-pyrazol-5- | 5 mg |
| yl)(phenyl)methoxy-N-methylethanamine | |
| 0.1 N Sodium hydroxide | c.s. pH 6 |
| Water for injection c.s.p. | 0.1 ml |

### EXAMPLE 5

Example of a Formulation (A) for a Tablet

| | |
|---|---|
| 2-((1-methyl-1H-pyrazol-5- | 50 mg |
| yl)(phenyl)methoxy-N-methylethanamine | |
| Sodium croscarmelose (Ac-Di-Sol) | 3.2 mg |
| Collodial silica dioxide (Aerosyl 200) | 0.8 mg |
| Magnesium stearate, NF | 1.6 mg |
| Providone K-30 | 4.0 mg |
| Microcrystalline cellulose (Avicel PH-102) | 14.6 mg |
| Lactose monohydrate (Farmatose 200 M) | 15.8 mg |
| Total | 90.0 mg |

### EXAMPLE 6

Example of a Formulation (B) for a Tablet

| | |
|---|---|
| 2-((1-methyl-1H-pyrazol-5- | 100 mg |
| yl)(phenyl)methoxy-N-methylethanamine | |
| Sodium croscarmelose (Ac-Di-Sol) | 16 mg |
| Collodial silica dioxide (Aerosyl 200) | 4 mg |
| Magnesium stearate, NF | 8 mg |
| Providone K-30 | 20 mg |
| Microcrystalline cellulose (Avicel PH-102) | 123 mg |
| Lactose monohydrate (Farmatose 200 M) | 129 mg |
| Total | 400 mg |

### EXAMPLE 6

Example of a Formulation (C) for a Tablet

| | |
|---|---|
| 2-((1-methyl-1H-pyrazol-5- | 200 mg |
| yl)(phenyl)methoxy-N-methylethanamine | |
| Sodium croscarmelose (Ac-Di-Sol) | 17.5 mg |
| Collodial silica dioxide (Aerosyl 200) | 1.5 mg |
| Sodium stearic fumarate | 6 mg |
| Polyethylene glycol 8000 | 15 mg |
| Microcrystalline cellulose (Avicel PH-102) | 37.5 mg |
| Lactose monohydrate (Farmatose 200 M) | 22.5 mg |
| Total | 300 mg |

### EXAMPLE 8

Example of a Formulation (C) for a Tablet

| | |
|---|---|
| 2-((1-methyl-1H-pyrazol-5- | 20 mg |
| yl)(phenyl)methoxy-N-methylethanamine | |
| Sodium croscarmelose (Ac-Di-Sol) | 1.75 mg |
| Collodial silica dioxide (Aerosyl 200) | 0.15 mg |
| Sodium stearic fumarate | 0.6 mg |
| Polyethylene glycol 8000 | 1.5 mg |
| Microcrystalline cellulose (Avicel PH-102) | 3.75 mg |
| Lactose monohydrate (Farmatose 200 M) | 2.25 mg |
| Total | 30 mg |

### EXAMPLE 9

Example of a Formulation of a Capsule

| | |
|---|---|
| 2-((1-methyl-1H-pyrazol-5- | 20.0 mg |
| yl)(phenyl)methoxy-N-methylethanamine | |
| Collodial silica dioxide | 0.08 mg |
| Magnesium stearate | 0.24 mg |
| Lactose | 27.68 mg |
| Total | 48.0 mg |

### EXAMPLE 10

### a) Synthesis of phenyl 2-((1-methyl-1H-pyrazol-5-yl)(phenyl)methoxy)ethylmethylcarbamate

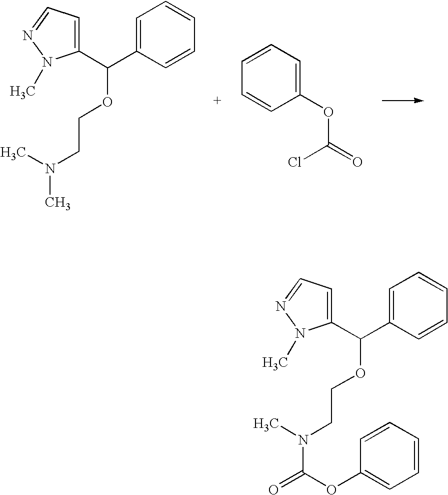

A solution of phenyl chloroformate (2.55 g, 1.63 mmol) in dichloromethane 10 mL was added dropwise to a mixture of 2-((1-methyl-1H-pyrazol-5-yl)(phenyl)methoxy)-N,N-dimethylethanamine (3.85 g, 14.85 mmol) (described in EP 289380 and U.S. Pat. No. 5,017,596) and potassium carbonate (4.4 g, 32.1 mmol) in dichloromethane (50 mL). The mixture was stirred 4 days at room temperature. Then the solid was filtered off, and the organic layer was washed with a diluted NaOH solution and water, dried over anhydrous sodium sulphate and concentrated. The residue was purified by silicagel column chromoatography (eluant CHCl₃/MeOH=98:2) to afford 4.4 g (81.5%) of phenyl 2-((1-methyl-1H-pyrazol-5-yl)(phenyl)methoxy)ethylmethylcarbamate as yellow oil.

NMR ¹H (CDC1₃) δ: 3.07 (s, CH₃—N, 3H), 3.18 (s, CH₃—N, 3H), 3.61 (m, —CH₂—N, 2H), 3.69 (m, —CH₂—N, —CH₂—O, 6H), 3.74 (s, CH₃—N, 3H), 3.76 (s, CH₃—N, 3H), 5.52 (s, 2H), 6.07 (d, J=1.5 Hz, 2H), 6.96 (m, 2H), 7.09 (m, 2H), 7.19 (m, 2H), 7.30-7.42 (m, 16H).

### b) Synthesis of 2-((1-metyl-1H-pyrazol-5-yl)(phenyl)methoxy)-N-methylethanamine

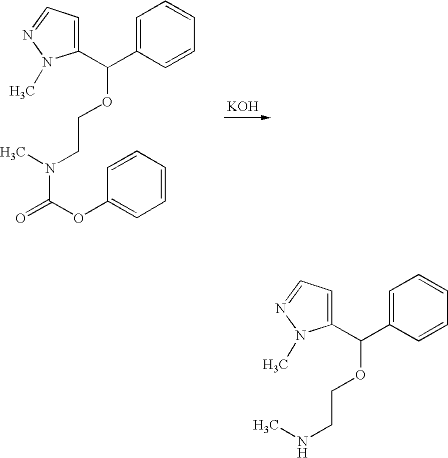

A solution of phenyl 2-((1-methyl-1H-pyrazol-5-yl)(phenyl)methoxy)ethylmethylcarbamate (4.4 g, 12.05 mmol) (prepared according to a)) and KOH 85% (10.2 g, 155 mmoles) in ethylene glycol (60 mL) was heated 1 h at 160° C. Then, the mixture was poured into 200 g ice and extracted with ethyl ether. The organic layer was separated, washed with water and brine, dried over anhydrous sodium sulphate and concentrated. The residue was purified by siliagel column chromatography (eluant, CHCl₃/MeOH=9:1) to afford 2.1 g (71.2%) of 2-((1-methyl-1H-pyrazol-5-yl)(phenyl)methoxy)-N-methylethanamine as a yellow oil.

IR (film): 2937, 2866, 2850, 1451, 1089, 1071, 782, 747, 726 cm⁻¹.

NMR ¹H (CDC1₃) δ: 1.69 (b.b., 1H), 2.42 (s, 3H), 2.78 (t, J=5.2 Hz, 2H), 3.58 (t, J=5.2 Hz, 2H), 3.77 (s, 3H), 5.46 (s, 1H), 5.95 (d, J=1.9 Hz, 1H), 7.25-7.40 (m, 6H).

### EXAMPLE 11

### Synthesis of 2-((1-methyl-1H-pyrazol-5-yl)(phenyl)methoxy)-N-methylethanamine citrate

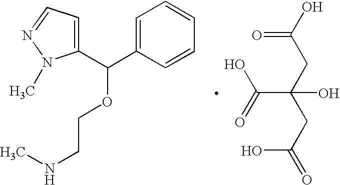

A solution of citric acid monohydrate (1.22 g, 5.8 mmol) in abs. ethanol (6 mL) was added to a solution of 2-((1-methyl-1H-pyrazol-5-yl)(phenyl)methoxy)-N-methylethanamine (1.3 g, 5.3 mmol) in abs ethanol 5 mL. The mixture was stirred for 1 h. The solvents were removed in vacuo and the residue redissolved and crystallized from isopropanol-ether to yield 2.05 g (89.1%) of 2-((1-methyl-1H-pyrazol-5-yl)(phenyl)methoxy)-N-methylethanamine citrate.

m.p. .61-62° C.

IR (KBr): 3680-2390 (b.b.), 1722, 1609, 1399, 1202, 1103 cm⁻¹.

NMR ¹H (DMSO-d₆) δ: 2.42-2.64 (m, 8H) 3.16 (m, 2H), 3.57 (m, 1H), 3.67 (m, 1H), 3.76 (s, 3H), 5.74 (s, 1H), 5.91 (d, J=1.5 Hz, 1H), 7.30 (d, J=1.5 Hz, 1H), 7.35-7.43 (m, 5H).

### EXAMPLE 12

### Synthesis of (R)-2-((1-methyl-1H-pyrazol-5-yl)(phenyl)methoxy)-N-methylethanamine

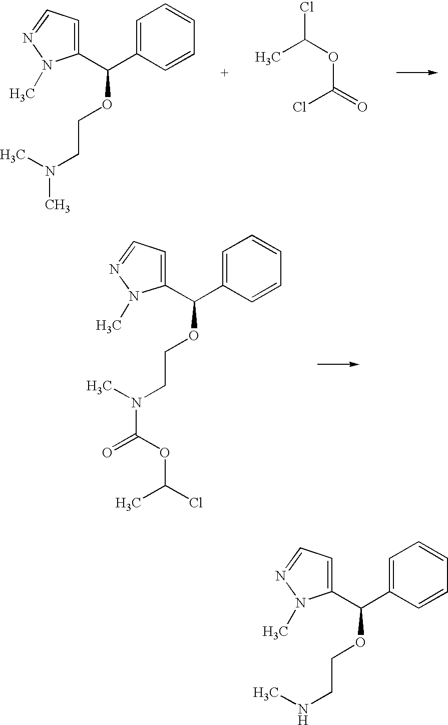

A solution of 1-chloroethyl chloroformate (1.81 g, 12.7 mmol) in methylene chloride (10 mL) was added dropwise to a suspension of 2-(R)-(1-methyl-1H-pyrazol-5-yl)(phenyl)methoxy)-N,N-dimethylethanamine (3 g, 11.6 mmol) (prepared as described in WO 99/0250b or WO 99/07684) and potassium carbonate (3.5 g, 25.1 mmol) in methylene chloride (50 mL). The mixture was stirred at room temperature for 3 days. Then, the solid was filtered off, and the organic layer was washed with a diluted NaOH solution and water, dried over anhydrous sodium sulfate and concentrated. The residue was purified by silicagel column chromatography (eluant, CHCl₃/AcOEt=1:1) to afford 2.1 g (53%) of the diastereomeric mixture of 1-chloroethyl 2-((R)-1-methyl-1H-pyrazol-5-yl)phenyl)methoxy)ethylmethylcarbamate as a yellow oil.

A solution of the diastereomeric mixture of 1-chloroethyl 2-((R)-1-methyl-1H-pyrazol-5-yl)phenyl)methoxy)ethylmethylcarbamate (2.1 g, 6.0 mmol) in MeOH (50 mL) was refluxed for 12 h. The mixture was concentrated in vacuo and the residue was purified by silicagel column chromatography (eluant, CHCl₃/MeOH=9:1) to afford 1.6 g of 2-((R)-1-methyl-1H-pyrazol-5-yl)phenyl)methoxy)-N-methylethanamine hydrochloride. Then the hydrochloride compound was dissolved in water, basified with dilute NaOH solution and extracted with CHCl₃. The organic layer was separated, dried over anhydrous sodium sulfate and concentrated to dryness to yield 1.22 g (83%) of 2-((R)-1-nethyl-1H-pyrazzol-5-yl)(phenyl)methoxy)-N-methylethanamine as a yellow oil.

IR (film): 2931, 2853, 1451, 1088, 1071, 782, 746, 725, 703 cm⁻¹.

NMR 1H (CDC1₃) δ: 1.66 (b.b., 1H), 2.42 (s, 3H), 2.78 (t, J=5.2 Hz, 2H), 3.58 (t, J=5.2 Hz, 2H), 3.76 (s, 3H), 5.45 (s, 1H), 5.97 (s, 1H), 7.25-7.40 (m, 6H).

[α]_{D}²⁰+29.7 (c=1.0, MeOH)

### EXAMPLE 13

### Synthesis of 2-((R)-(1-methyl-1H-pyrazol-5-yl)(phenyl)methoxy)-N-methylethanamine oxalate

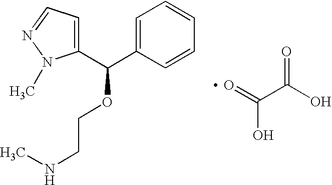

A solution of oxalic acid dihydrate (620 mg 4.9 mmoles) in abs. ethanol (4 ml) was added at room temperature to a solution of 2-((R)-(1-methyl-1H-pyrazol-5-yl)(phenyl)methoxy)-N-methylethanamine (1.2 g 4.9 mmoles) in abs. ethanol (6 ml). The mixture was stirred until precipitation was completed. The solid obtainedwas filtered and cristallized from ethanol-ether to yield 1.35 g (89.1%) of 2-((R)-(1-methyl-1H-pyrazol-5-yl)(phenyl)methoxy)-N-methylethanamine oxalate as a white solid.

m.p. of.: 141-142° C.

¹H NMR (DMSO-d₆) δ: 2.54 (s, 3H), 3.58 (m, 2H), 3.66 (m, 2H), 3.75 (s, 3H), 5.74 (s, 1H), 5.91 ( s, 1H), 7.30-7.42 (ca, 6H).

[α]_{D}²⁰+20.6 (c=1.0, MeOH)

### EXAMPLE 14

### Synthesis of 2-((S)-(1-methyl-1H-pyrazol-5-yl)(phenyl)methoxy)-N-methylethanamine

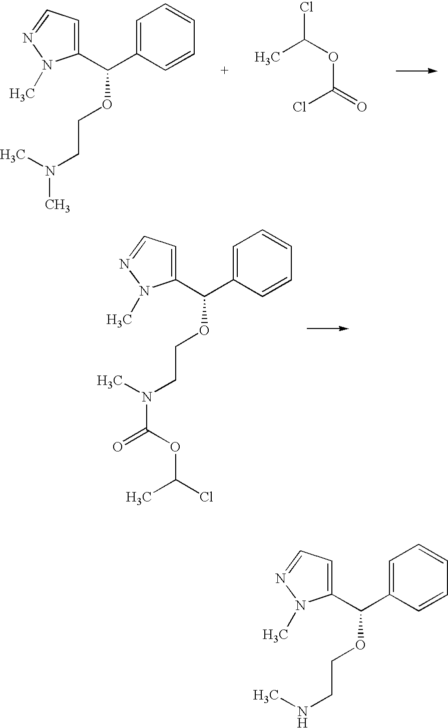

A solution of 1-chloroethyl chloroformate (1.04 g, 7.3 mmoles) in methylene chloride (10 ml) was added dropwise to a suspension of 2-((S)-(1-methyl-1H-pyrazol-5-yl)(phenyl)methoxy)-N,N-dimethyl ethanamine (1.72 g, 6.6 mmoles) (prepared as described in WO 99/02500 or WO 99/07684) and potassium carbonate (2.0 g, 14.5 mmoles) in methylene chloride (50 ml). The mixture was stirred at room temperature for 3 days. Then the solid was filtered off, and the organic layer was washed with a diluted NaOH solution and water, dried over anhydrous sodium sulphate and concentrated. The residue was purified by silicagel column chromatography (eluant, CHCl₃/AcOEt=1:1) to afford 1.1 g (48%) of the diastereomeric mixture of 1-chloroethyl 2-((S)-(1-methyl-1H-pyrazol-5-yl)(phenyl)methoxy)ethylmethylcarbamate as a yellow oil.

A solution of the diastereomeric mixture of 1-chloroethyl 2-((S)-(1-methyl-1H-pyrazol-5-yl)(phenyl)methoxy)ethylmethylcarbamate (1.1 g, 3.1 mmoles) in MeOH (25 ml) was refluxed for 12 h. The mixture was concentrated in vacuoand the residue was purified by silicagel column chromatography (eluant CHCl₃/MeOH=9:1) to afford 0.78 g of 2-((S)-(1-methyl-1H-pyrazol-5-yl)(phenyl)methoxy)-N-methylethanamine hydrochloride. Then the hydrochloride compoundwas dissolved in water, basified with diluted NaOH solution and extracted with CHCl₃. The organic layer was separated, dried over anhydrous sodium sulphate and concentrated to dryness to yield 0.54 g (70%) of 2-((S)-(1-methyl-1H-pyrazol-5-yl)(phenyl)methoxy)-N-methylethanamine as a yellow oil.

IR (film): 2940, 2866, 1451, 1088, 1071, 783, 747, 725, 703 cm⁻¹.

¹H NMR (CDC1₃) δ: 2.44 (s, 3H), 2.79 (t, J=5.2 Hz, 2H), 3.59 (t, J=5.2 Hz, 2H), 3.78 (s, 3H), 5.47 (s, 1H), 5.97 (d, J=1.9 Hz, 1H), 7.25-7.39 (m, 6H).

[α]_{D}²⁰⁻³¹ (c=1.0, MeOH)

### EXAMPLE 15

### Synthesis of 2-((S)-(lmethyl-1H-pyrazol-5-yl)(phenyl)methoxy)-N-methylethanamine oxalate

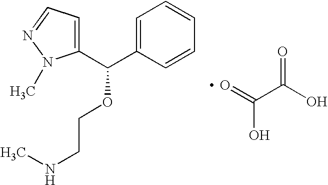

A solution of oxalic acid dehydrate (272 mg 2.1 mmoles) in abs ethanol (2 ml)was added at room temperature to a solution of 2-((S)-(1-methyl-1H-pyrazol-5-yl)(phenyl)methoxy)-N-methylethanamine (0.53 g, 2.1 mmoles) in abs ethanol (3 ml). The mixture was stirred until precipitation was completed. The solid obtained was filterd and crystallized from ethanol-ether to yield 0.53 g (73.3%) of 2-((S)-(lmethyl-1H-pyrazol-5-yl)(phenyl)methoxy)-N-methylethanamine oxalate as a white solid

m.p.: 142-143° C.

¹H NMR (DMSO-d₆) δ: 2.54 (s, 3H), 3.58 (m, 2H), 3.66 (m, 2H), 3.75 (s, 3H), 5.74 (s, 1H), 5.91 ( s, 1H), 7.30-7.42 (m, 6H).

[α]_{D}²⁰−19.1 (c=1.0, MeOH).

### EXAMPLE 16

### General Production of Compounds According to Formulas (I), (II), (III) or (IV)

1 g of 2-((1-methyl-1H-pyrazol-5-yl) (phenyl)methoxy)-N,N-dimethylethanamine is soluted in TRIS/HCL-Buffer pH 7.4. MgCl as well as other known cofactors of CytP450 are added and the solution is incubated for 2 hours with CytP450 3A4 and CytP450 2D6 at 37° C. Following that, the solution is separated using HPLC and the metabolites are isolated.

### EXAMPLE 17

### Physiological Tests

Antidepressant Activity

The antidepressant activity of compound 1 has been studied in different test. First, of all it has been shown that compound 1 has affinity for serotonin transporter receptor like other antidepressants. The antidepressant activity of compound 1 has also been shown in 3 experimental tests in vivo: test of inhibition of reserpine-induced ptosis in mice, test of inhibition of aversive situation-induced immobility in mice and water despair test in rats.

Serotonin Transporter Affinity.

This affinity was studied in rat brain cortex, using (3H)paroxetine (0.05 nm) as radioligand and 5-Hydroxytriptamine (serotonin)(100 μM) as non specific ligand. Compound 1 had a Ki=116 nM, which could be considered enough to support a significant antidepressant activity.

Test of Inhibition of Reserpin-induced Ptosis in Mice.

It has been used the test described by S.Garattini et al. (Med.Exp. 1960, 3: 315-320). The antidepressant drugs inhibit the parpebral ptosis induced by reserpine (25 mg/Kg, i.p.) in mice. Compound 1 showed a clear antidepressant activity, which was in a similar range to other antidepressants, like imipramine or sertraline. Its ED-50 was14 mg/kg, po (Table 1)

Test of Inhibition of Immobility Induced by an Aversive Situatuion in Mice

The test was described by R. D. Porsolt et al. (Arch.int.Pharmacodyn. 1987, 288: 11-30). Following suchg method, the compound to be studied was administered by ip route, and 30 minutes later mice were submitted to an aversive situation: they were tail suspended in the itematic-TST for 6 minutes. Mobility time and movements power were authomatically measured and registered in the above mentioned device. The potential antidepressant reduce the mice immobility time. Compound 1 had a similar activity to that of imipramine and sertraline. Its ED-50 was17 mg/Kg, i.p. (Table 1).

Water Despair Test in Rats

The test was described by R. D. Porsolt et al. (Europ.J.Pharmacol. 1978, 47:379-391). Rats were let to swim in a water (28-30° C.) filled cylinder for a period of 15 minutes. The following day, 24 hours later, were let to swim in the water, but only for 5 minutes, and it was registerd the immobility time. The compounds to be studied were administered by ip route, 60 minutes before the test. The antidepressants reduce the immobility time. Compound 1, also in this test, showed a clear antidepressant activity, similar to that of sertraline and slightly higher than that of desipramine. Its ED50 was 33 mg/Kg, i.p. (Tablel).

TABLE 1
Antidepressant activity in vivo.

| | Inhibition | Immobility | | |
|---|---|---|---|---|
| | test Reserpin | test Tail | Swimming Test | |
| | ptosis In | hanging In | Forced swimming | |
| | mice ED50 | mice ED50 | In rats | |
| Product | (mg/Kg, p.o.) | (mg/Kg, i.p.) | ED50 (mg/Kg, i.p.) | |
| Imipramine | 7 | 7 | — | |
| Desipramine | — | — | I = 32% | (30 mg/Kg) |
| Sertraline | 17 | 8 | 28 | |
| Compound 1 | 14 | 17 | 33 | |
| Cizolirtine | 26 | 17 | inactive | (40 mg/Kg) |
| Compound 2 | inactive | 33 | inactive | (40 mg/Kg) |
| | (40 mg/Kg) | | | |

Compound 1:
2-((1-methyl-1H-pyrazol-5-yl)(phenyl)methoxy)-N-methylethanamine
   Compound 2:
2-((1-methyl-1H-pyrazol-5-yl)(thiophen-2-yl)methoxy)-N-methylethanamine; oxalate

Aditionally the compound 1, accoridng example 1, and its enantiomers were active in rat urinary incontinence test. They inhibited the amplitude of the rat bladder contractions. The compounds were evaluated in a model of cystometry in the anesthesized rat, looking for the effects on isovolumetric rhythmic bladder contractions. The method, which evaluatesthe effects of the test substances on lower urinary tract function, followed that described by Y. Kimura et al. ( Int. J. Urol. 1996, 3: 218-227).

Activity Against Urinary Incontinence

The activity against urinary incontinence of cizolirtine is demonstrated by urodinamic evaluation in vivo (cystometry). This is performed on Sprague-Dawley male and female anaesthetized rats. Left carotid artery is inserted with a catheter for the continuous measure of systemic arterial pressure and cardiac frequency. After an incision to the abdomen, a catheter is inserted on the bladder and connected to a pressure transducer and to an infusion pump. The urethra is ligated and the bladder is voided. After stabilization period, saline solution is infused (0.9 % NaC1solution at 5 ml/h rate) and the basal parameters of intravesical pressure are measured for 20 min. Cystometry parameters are the infusion volume and pressure needed to activate the micturition reflex, and the micturition frequency. After stabilization period, the compounds are administered i.v. and, 15 min. later, saline is infused and urodynamic parameters are measured again to compare with basal.

Effect On Cystometries

Cizolirtine (10 mg/kg; i.v) significantly increased mean micturition volume and significantly reduced the bladder pressure. In addition, it shows trends to decrease micturition frequency and to augment the volume required for first micturition, with no alteration of bladder contractility.

Further the effect on acetic acid-induced bladder hiperactivity is tested. The infusion of acetic acid (0.3% v/v) caused a significant increase of micturition frequency and a significant reduction of micturition volume. These alterations are significantly reduced by acute administration of cizolirtine (10 mg/kg; i.v.). Acetic acid infusion also increased bladder pressure, an effect that was reversed by cizolirtine. Neither acetic acid nor cizolirtine significantly affected contractile capacity of the bladder.

This application also discloses preferred aryl or heteroaryl azolylcarbinole derivatives of general formula (I), (II), (III), (IV) in which:
R¹ represents a hydrogen atom or a lower alkyl group from C₁ to C₄;
R² represents a phenyl radical or a thienyl radical, with no substitutions or optionally with 1, 2 or 3 equal or different substituents, selected from the group comprised of hydroxy, fluoride, chloride, bromide, methyl, trifluoromethyl and methoxy, or —O-(glucoronic acid);
R³ represents a five-armed nitrogenated aromatic heterocycle that contains one to three nitrogen atoms, without substitutions or optionally substituted by 1 or 2 equal or different substituents selected from a group comprised by fluoride, chloride, bromide and methyl;
R⁴ represents H or C₁₋₄-Alkyl;
R⁵ represents H or C₁₋₄-Alkyl;
R⁹ is glucoronic acid;
R¹⁰ represents C₁₋₄-Alkyl; and
n is 1 to 4 or p is 1 to 3;
optionally in the form of its racemate, pure stereoisomers, especially enantiomers or diastereomers or in the form of mixtures of stereoisomers, especially enantiomers or diastereomers, in any suitable ratio; in the form shown or in form of the acid or base or in form of a salt, especially a physiologically acceptable salt, or in form of a solvate, especially a hydrate;
with the proviso that when R² is a phenyl group unsubstituted or substituted with 1, 2 or 3 equal or different substitutents selected from fluoride, chloride bromide, trifluoromethyl or methoxy, R⁴ is not H or Cl alkyl within formula I and R⁴ or R⁵ are not Cl alkyl within formula II.

This application also provides an aryl or heteroaryl azolylcarbinole derivative of general formula (VIII) in which:
R¹ represents a hydrogen atom or a lower alkyl group from C₁ to C₄;
R² represents a phenyl radical or a thienyl radical, with no substitutions or optionally with 1, 2 or 3 equal or different substituents, selected from the group comprised of hydroxy, fluoride, chloride, bromide, methyl, trifluoromethyl and methoxy, or —O-(glucoronic acid);
R³ represents a five-armed nitrogenated aromatic heterocycle that contains one to three nitrogen atoms, without substitutions or optionally substituted by 1 or 2 equal or different substituents selected from a group comprised by fluoride, chloride, bromide and methyl;
R⁴ represents H or C₁₋₄-Alkyl;
R⁵ represents H or C₁₋₄-Alkyl;
R⁹ is glucoronic acid;
R¹⁰ represents C₁₋₄-Alkyl;
n is 1 to 4 or p is 1 to 3;
and Z has the same meanings as already mentioned;
optionally in the form of its racemate, pure stereoisomers, especially enantiomers or diastereomers or in the form of mixtures of stereoisomers, especially enantiomers or diastereomers, in any suitable ratio; in the form shown or in form of the acid or base or in form of a salt, especially a physiologically acceptable salt, or in form of a solvate, especially a hydrate;
with the proviso that when R² is a phenyl group unsubstituted or substituted with 1, 2 or 3 equal or different substitutents selected from, fluoride, chloride, bromide, trifluoromethyl or methoxy and Z represents: 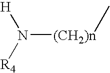
R⁴ is not H or C1 alkyl,
and with the proviso that when R² is a phenyl group unsubstituted or substituted with 1, 2 or 3 equal or different substitutents selected from, fluoride, chloride, bromide, trifluoromethyl or methoxy and Z represents: 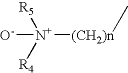
R⁴ or R⁵ are not C1alkyl.

Also provided and currently preferred is an aryl or heteroaryl azolylcarbinole derivative of general formula (I), (II), (III) or (IV) in which R₁ is selected from hydrogen or from a group consisting of methyl, ethyl, propyl, isopropyl, butyl, isobutyl, sec-butyl and tert-butyl.

Further provided and currently preferred is an aryl or heteroaryl azolylcarbinole derivative of general formula (I) and (II) in which R₄ is selected from hydrogen, methyl or ethyl, especially methyl or hydrogen.

Still further provided and currently preferred is an aryl or heteroaryl azolylcarbinole derivative of general formula (II) in which R₅ is selected from hydrogen, methyl or ethyl, especially methyl or hydrogen.

Moreover, this application provides and currently preferred is an aryl or heteroaryl azolylcarbinole derivative of general formula (I), (II), (III) or (IV), in which R³ is selected from 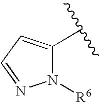
with R⁶ being selected from hydrogen, fluoride, chloride, bromide and methyl. There it is preferred if R⁶ is hydrogen or methyl.

Also provided and currently preferred is an aryl or heteroaryl azolylcarbinole derivative of general formula (I), (II), (III) or (IV) in which R² is selected from: 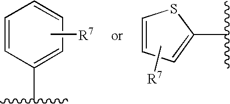
with R⁷ being selected from the group consisting of hydroxyl, hydrogen, fluoride, chloride, bromide, methyl, trifluoromethyl and methoxy, or —O-(glucoronic acid).

There it is preferred that R⁷ is hydrogen or hydroxyl, or —O-(glucoronic acid).

Further provided and currently highly preferred is an aryl or heteroaryl azolylcarbinole derivative of general formula (I) or (II) in which the compound is a compound according to one of the general formulas (Ia) or (IIa) 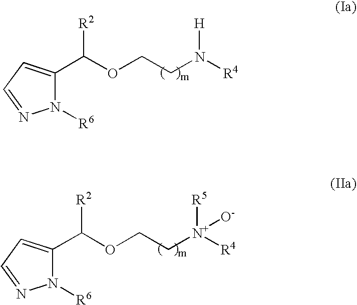
in which
m is 1 or 2;
R² represents a phenyl radical or a thienyl radical, with no substitutions or optionally with 1, 2 or 3 equal or different substituents, selected from the group comprised of hydroxyl, fluoride, chloride, bromide, methyl, trifluoromethyl and methoxy;
R⁴ represents H or C₁₋₄-Alkyl;
R⁵ represents H or C₁₋₄-Alkyl; and
R⁶ is selected from hydrogen, fluoride, chloride, bromide and methyl.

Further provided and currently preferred is an aryl or heteroaryl azolylcarbinole derivative of general formula (Ia) or (IIa) in which
R² is selected from: 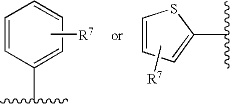
   with R₇ being selected from the group consisting of hydroxyl, hydrogen, fluoride, chloride, bromide, methyl, trifluoromethyl and methoxy.

There it is preferred that R⁷ is hydrogen or hydroxyl.

Still further provided and currently preferred is an aryl or heteroaryl azolylcarbinole derivative of general formula (Ia) or (IIa) in which R⁴ is methyl or ethyl, preferably methyl.

Also provided and currently preferred is an aryl or heteroaryl azolylcarbinole derivative of general formula (IIa), in which R⁵ is hydrogen, methyl or ethyl, preferably hydrogen or methyl.

Further provided and currently preferred is an aryl or heteroaryl azolylcarbinole derivative of general formula (Ia) or (IIa) in which R⁶ is hydrogen or methyl.

Moreover, this application further provides and currently highly preferred is an aryl or heteroaryl azolylcarbinole derivative of general formula (I) or (II) in which the compound is a compound according to one of the general formulas (Ib), (Ic), (IIb) or (IIc) 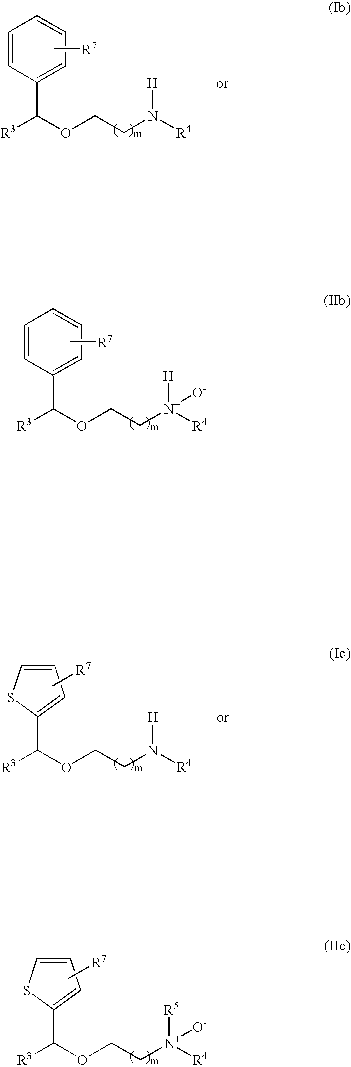
in which
m is 1 or 2;
R³ represents a five-armed nitrogenated aromatic heterocycle that contains one to three nitrogen atoms, without substitutions or optionally substituted by 1 or 2 equal or different substituents selected from a group comprised by fluoride, chloride, bromide and methyl;
R⁴ represents H or C₁₋₄-Alkyl;
R⁵ represents H or C₁₋₄-Alkyl; and
R⁷ is selected from the group consisting of hydroxyl, hydrogen, fluoride, chloride, bromide, methyl, trifluoromethyl and methoxy.

Also provided and currently preferred is an aryl or heteroaryl azolylcarbinole derivative of general formula (Ib), (Ic), (IIb) or (IIc), in which R⁷ is hydrogen or hydroxyl.

Further provided and currently preferred is an aryl or heteroaryl azolylcarbinole derivative of general formula (Ib), (Ic), (IIb) or (IIc), in which R³ is selected from 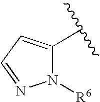
with R⁶ being selected from hydrogen, fluoride, chloride, bromide and methyl. There it is preferred if R⁶ is hydrogen or methyl.

Still further provided and currently preferred is an aryl or heteroaryl azolylcarbinole derivative of general formula (Ib), (Ic), (IIb) or (IIc), in which R⁴ is methyl or ethyl, preferably methyl.

Also provided and currently preferred is an aryl or heteroaryl azolylcarbinole derivative of general formula (IIb) or (IIc), in which R⁵ is hydrogen, methyl or ethyl, preferably hydrogen or methyl.

Moreover, this application provides and currently highly preferred is an aryl or heteroaryl azolylcarbinole derivative of general formula (I) or (II) in which the compound is a compound according to one of the general formulas (Id), (Ie), (IId) or (IIe) 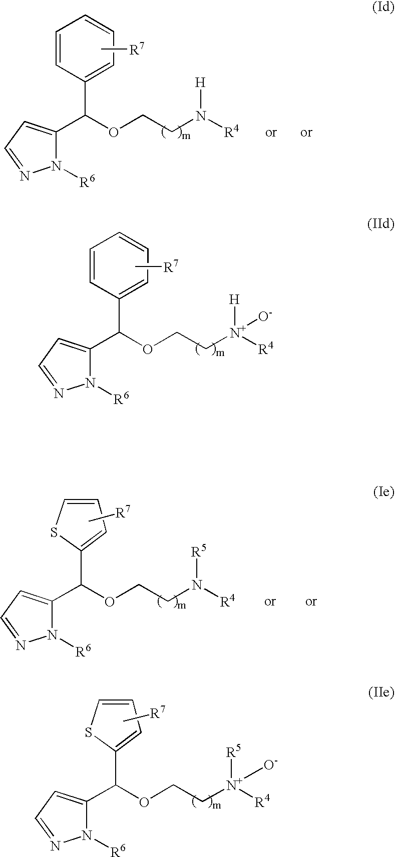
in which
m is 1 or 2;
R⁴ is selected from H or lower C₍₁₋₄₎-Alkyl;
R⁵ is selected from H or lower C₍₁₋₄₎-Alkyl;
R⁶ is selected from hydrogen, fluoride, chloride, bromide and methyl; and
R₇ is selected from the group consisting of hydroxyl, hydrogen, fluoride, chloride, bromide, methyl, trifluoromethyl and methoxy.

Also provided and currently preferred is an aryl or heteroaryl azolylcarbinole derivative of general formula (Id), (Ie), (IId) or (IIe), in which R⁷ is hydrogen or hydroxyl.

Further provided and currently preferred is an aryl or heteroaryl azolylcarbinole derivative of general formula (Id), (Ie), (IId) or (IIe), in which R⁶ is hydrogen or methyl.

Still further provided and currently preferred is an aryl or heteroaryl azolylcarbinole derivative of general formula (Id), (Ie), (IId) or (IIe), in which R⁴ is methyl or ethyl, preferably methyl.

Also provided and currently preferred is an aryl or heteroaryl azolylcarbinole derivative of general formula (IId) or (IIe), in which R⁵ is hydrogen, methyl or ethyl, preferably hydrogen or methyl.

Further provided and currently preferred are the salts, the physiologically acceptable salts of the new compounds. In this regards it is highly preferred that the compounds according to general formula (II) are in a form according to general formula (V) 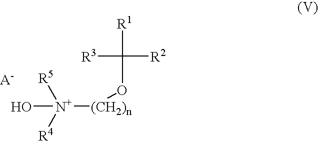
in which
A⁻ represents an anion;
R¹ represents a hydrogen atom or a lower alkyl group from C₁ to C₄;
R² represents a phenyl radical or a thienyl radical, with no substitutions or optionally with 1, 2 or 3 equal or different substituents, selected from the group comprised of hydroxyl, fluoride, chloride, bromide, methyl, trifluoromethyl and methoxy;
R³ represents a five-armed nitrogenated aromatic heterocycle that contains one to three nitrogen atoms, without substitutions or optionally substituted by 1 or 2 equal or different substituents selected from a group comprised by fluoride, chloride, bromide and methyl;
R⁴ represents H or C₁₋₄-Alkyl;
R⁵ represents H or C₁₋₄-Alkyl.

It is also highly preferred if the new aryl or heteroaryl azolylcarbinole derivatives according to the application are selected from:
4-((2-(dimethylamino)ethoxy)(1-methyl-1H-pyrazol-5-yl)methyl)phenol;
(±)-4-((2-(dimethylamino)ethoxy)(1-methyl-1H-pyrazol-5-yl)methyl)phenol;
(+)-4-((2-(dimethylamino)ethoxy)(1-methyl-1H-pyrazol-5-yl)methyl)phenol;
(−)-4-((2-(dimethylamino)ethoxy)(1-methyl-1H-pyrazol-5-yl)methyl)phenol;
6-(4-((2-(dimethylamino)ethoxy)(1-methyl-1H-pyrazol-5-yl)methyl)phenoxy)-tetrahydro-3,4,5-trihydroxy-2H-pyran-2-carboxylic acid
(±)-6-(4-((2-(dimethylamino)ethoxy)(1-methyl-1H-pyrazol -5-yl)methyl)phenoxy)-tetrahydro-3,4,5-trihydroxy -2H-pyran-2-carboxylic acid
(+)-6-(4-((2-(dimethylamino)ethoxy)(1-methyl-1H-pyrazol -5-yl)methyl)phenoxy)-tetrahydro-3,4,5-trihydroxy -2H-pyran-2-carboxylic acid
(−)-6-(4-((2-(dimethylamino)ethoxy)(1-methyl-1H-pyrazol -5-yl)methyl)phenoxy)-tetrahydro-3,4,5-trihydroxy -2H-pyran-2-carboxylic acid
6-(4-((2-(methylamino)ethoxy)(1-methyl-1H-pyrazol-5-yl)methyl)phenoxy)-tetrahydro-3,4,5-trihydroxy-2H-pyran -2-carboxylic acid
(±)-(6-(4-((2-(methylamino)ethoxy)(1-methyl-1H-pyrazol -5-yl)methyl)phenoxy)-tetrahydro-3,4,5-trihydroxy-2H-pyran -2-carboxylic acid
(+)-6-(4-((2-(methylamino)ethoxy)(1-methyl-1H-pyrazol -5-yl)methyl)phenoxy)-tetrahydro-3,4,5-trihydroxy-2H-pyran -2-carboxylic acid
(−)-6-(4-((2-(methylamino)ethoxy)(1-methyl-1H-pyrazol -5-yl)methyl)phenoxy)-tetrahydro-3,4,5-trihydroxy-2H-pyran -2-carboxylic acid
6-(N-(2-((1-methyl-1H-pyrazol-5-yl)(phenyl)methoxy)ethyl)-N-methylamino)-tetrahydro -3,4,5-trihydroxy-2H-pyran-2-carboxylic acid
(±)-6-(N-(2-((1-methyl-1H-pyrazol-5-yl)(phenyl)methoxy)ethyl)-N-methylamino)-tetrahydro -3,4,5-trihydroxy-2H-pyran-2-carboxylic acid
(+)-6-(N-(2-((1-methyl-1H-pyrazol-5-yl)(phenyl)methoxy)ethyl)-N-methylamino)-tetrahydro -3,4,5-trihydroxy-2H-pyran-2-carboxylic acid
(−)-6-(N-(2-((1-methyl-1H-pyrazol-5-yl)(phenyl)methoxy)ethyl)-N-methylamino)-tetrahydro -3,4,5-trihydroxy-2H-pyran-2-carboxylic acid
2-((1-methyl-1H-pyrazol-5-yl)(thiophen-2-yl)methoxy)ethanamine;
(±)-2-((1-methyl-1H-pyrazol-5-yl)(thiophen-2-yl)methoxy)ethanamine;
(+)-2-((1-methyl-1H-pyrazol-5-yl)(thiophen-2-yl)methoxy)ethanamine;
(−)-2-((1-methyl-1H-pyrazol-5-yl)(thiophen-2-yl)methoxy)ethanamine;
2-((1H-pyrazol-5-yl)(thiophen-2-yl)methoxy)-N,N-dimethylethanamine;
(±)-2-((1H-pyrazol-5-yl)(thiophen-2-yl)methoxy)-N,N-dimethylethanamine;
(+)-2-((1H-pyrazol-5-yl)(thiophen-2-yl)methoxy)-N,N-dimethylethanamine;
(−)-2-((1H-pyrazol-5-yl)(thiophen-2-yl)methoxy)-N,N-dimethylethanamine;
5-((2-(dimethylamino)ethoxy)(1-methyl-1H-pyrazol-5-yl)methyl)thiophen -3-ol;
(±)-5-((2-(dimethylamino)ethoxy)(1-methyl-1H-pyrazol-5-yl)methyl)thiophen -3-ol;
(+)-5-((2-(dimethylamino)ethoxy)(1-methyl-1H-pyrazol-5-yl)methyl)thiophen -3-ol;
(−)-5-((2-(dimethylamino)ethoxy)(1-methyl-1H-pyrazol-5-yl)methyl)thiophen -3-ol;
5-((2-(dimethylamino)ethoxy)(1-methyl-1H-pyrazol-5-yl)methyl)thiophen -2-ol;
(±)-5-((2-(dimethylamino)ethoxy)(1-methyl-1H-pyrazol-5-yl)methyl)thiophen -2-ol;
(+)-5-((2-(dimethylamino)ethoxy)(1-methyl-1H-pyrazol-5-yl)methyl)thiophen -2-ol;
(−)-5-((2-(dimethylamino)ethoxy)(1-methyl-1H-pyrazol-5-yl)methyl)thiophen -2-ol;
2-((1-methyl-1H-pyrazol-5-yl)(thiophen-2-yl)methoxy)acetic acid;
(±)-2-((1-methyl-1H-pyrazol-5-yl)(thiophen-2-yl)methoxy)acetic acid;
(+)-2-((1-methyl-1H-pyrazol-5-yl)(thiophen-2-yl)methoxy)acetic acid;
(−)-2-((1-methyl-1H-pyrazol-5-yl)(thiophen-2-yl)methoxy)acetic acid;
6-((5-((2-(dimethylamino)ethoxy)(1-methyl-1H-pyrazol-5-yl)methyl)thiophen -3-yl)methyl)-tetrahydro-3,4,5-trihydroxy -2H-pyran-2-carboxylic acid
(±)-6-((5-((2-(dimethylamino)ethoxy)(1-methyl-1H-pyrazol -5-yl)methyl)thiophen-3-yl)methyl)-tetrahydro -3,4,5-trihydroxy-2H-pyran-2-carboxylic acid
(+)-6-((5-((2-(dimethylamino)ethoxy)(1-methyl-1H-pyrazol -5-yl)methyl)thiophen-3-yl)methyl)-tetrahydro -3,4,5-trihydroxy-2H-pyran-2-carboxylic acid
(−)-6-((5-((2-(dimethylamino)ethoxy)(1-methyl-1H-pyrazol -5-yl)methyl)thiophen-3-yl)methyl)-tetrahydro -3,4,5-trihydroxy-2H-pyran-2-carboxylic acid
2-(methylamino)ethoxy)(1-methyl-1H-pyrazol-5-yl)methyl)thiophen -3-yl)methyl)-tetrahydro-3,4,5-trihydroxy -2H-pyran-2-carboxylic acid
(±)-2-(methylamino)ethoxy)(1-methyl-1H-pyrazol-5-yl)methyl)thiophen -3-yl)methyl)-tetrahydro-3,4,5-trihydroxy -2H-pyran-2-carboxylic acid
(+)-2-(methylamino)ethoxy)(1-methyl-1H-pyrazol-5-yl)methyl)thiophen -3-yl)methyl)-tetrahydro-3,4,5-trihydroxy -2H-pyran-2-carboxylic acid
(−)-2-(methylamino)ethoxy)(1-methyl-1H-pyrazol-5-yl)methyl)thiophen -3-yl)methyl)-tetrahydro-3,4,5-trihydroxy -2H-pyran-2-carboxylic acid
6-(N-(2-((1-methyl-1H-pyrazol-5-yl)(thiophen-2-yl)methoxy)ethyl)-N-methylamino)-tetrahydro -3,4,5-trihydroxy -2H-pyran-2-carboxylic acid
(±)-6-(N-(2-((1-methyl-1H-pyrazol-5-yl)(thiophen-2-yl)methoxy)ethyl)-N-methylamino)-tetrahydro -3,4,5-trihydroxy -2H-pyran-2-carboxylic acid
(+)-6-(N-(2-((1-methyl-1H-pyrazol-5-yl)(thiophen-2-yl)methoxy)ethyl)-N-methylamino)-tetrahydro -3,4,5-trihydroxy -2H-pyran-2-carboxylic acid
(−)-6-(N-(2-((1-methyl-1H-pyrazol-5-yl)(thiophen-2-yl)methoxy)ethyl)-N-methylamino)-tetrahydro -3,4,5-trihydroxy -2H-pyran-2-carboxylic acid;
   optionally (if applicable) also in form of a corresponding N-oxide according to any of formulas (II), (IIa), (IIb), (IIc), (IId) or (IIe); and optionally in form of a salt, especially a physiologically acceptable salt, most preferably the citrate or oxalate or in form of a solvate, especially a hydrate; and optionally (if applicable) also in form of a corresponding salt of an N-oxide according to formula (V).

Also highly preferred compounds are the enantiomers of the compounds described only as metabolites of the racemate in L. Martinez et al., “Absorption, distribution, metabolism and excretion of cizolirtine, a new analgesic compound, in rat and dog,” Xenobiotica, 1999 (29) 8, 859-871 or in Puig S., et al. J. Pharm. Biomed. Anal. “Validation of a chromatographic method to determine E-6006 and its metabolite E-6332 in rat and dog plasma by solid-phase extraction and capillary gas chromatography.” Application in pharmacokinetics, 24 (2001) 887-896 which are also highly active selected from:
(+)-2-((1-methyl-1H-pyrazol-5-yl)(phenyl)methoxy)-N-methylethanamine;
(−)-2-((1-methyl-1H-pyrazol-5-yl)(phenyl)methoxy)-N-methylethanamine;
(+)-N-oxo-2-((1-methyl-1H-pyrazol-5-yl)(phenyl)methoxy)-N,N-dimethylethanamine;
(−)-N-oxo-2-((1-methyl-1H-pyrazol-5-yl)(phenyl)methoxy)-N,N-dimethylethanamine;
(+)-2-((1-methyl-1H-pyrazol-5-yl)(phenyl)methoxy)acetic acid;
(−)-2-((1-methyl-1H-pyrazol-5-yl)(phenyl)methoxy)acetic acid;
(+)-2-((1-methyl-1H-pyrazol-5-yl)(phenyl)methoxy)ethanamine;
(−)-2-((1-methyl-1H-pyrazol-5-yl)(phenyl)methoxy)ethanamine;
(+)-N,N-dimethyl-2-(phenyl(1H-pyrazol-5-yl)methoxy)ethanamine;
(−)-N,N-dimethyl-2-(phenyl(1H-pyrazol-5-yl)methoxy)ethanamine;
(+)-2-((1-methyl-1H-pyrazol-5-yl)(thiophen-2-yl)methoxy)-N-methylethanamine;
(−)-2-((1-methyl-1H-pyrazol-5-yl)(thiophen-2-yl)methoxy)-N-methylethanamine;
   optionally in form of a salt, especially a physiologically acceptable salt, most preferably the citrate or oxalate or in form of a solvate, especially a hydrate.

Another aspect of the application is a combination of at least one aryl or heteroaryl azolylcarbinole derivative according to the application or one compound selected from
2-((1-methyl-1H-pyrazol-5-yl)(phenyl)methoxy)ethanamine,
2-((1-methyl-1H-pyrazol-5-yl)(phenyl)methoxy)-N-methylethanamine,
N-oxo-2-((1-methyl-1H-pyrazol-5-yl)(phenyl)methoxy)-N,N-dimethylethanamine;
2-((1-methyl-1H-pyrazol-5-yl)(phenyl)methoxy)acetic acid;
2-((1-methyl-1H-pyrazol-5-yl)(thiophen-2-yl)methoxy)-N-methylethanamine;
   optionally in the form of its racemate, pure stereoisomers, especially enantiomers or diastereomers or in the form of mixtures of stereoisomers, especially enantiomers or diastereomers, in any suitable ratio; also optionally in form of a salt, especially a physiologically acceptable salt, or in form of a solvate, especially a hydrate;
   and at least one compound of general formula VII 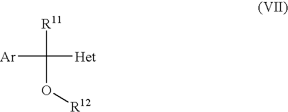
   in which
Ar represents a phenyl radical or a thienyl radical, with no substitutions or optionally with 1, 2 or 3 equal or different substituents, selected from the group comprised of fluoride, chloride, bromide, methyl, trifluoromethyl and methoxy; R₁₁ represents a hydrogen atom or a lower alkyl group from C₁ to C₄; R₁₂ represents a dialkyl(C₁-C₄) aminoalkyl (C₂-C₃), or azaheterocyclylalkyl (C₂-C₃) radical; and Het represents a five-armed nitrogenated aromatic heterocycle that contains one to three nitrogen atoms, without substitutions or optionally substituted by 1 or 2 equal or different substituents selected from a group comprised by fluoride, chloride, bromide and methyl;
   optionally in the form of its racemate, pure stereoisomers, especially enantiomers or diastereomers or in the form of mixtures of stereoisomers, especially enantiomers or diastereomers, in any suitable ratio;in the form shown or in form of the acid or base or in form of a salt, especially a physiologically acceptable salt, or in form of a solvate, especially a hydrate.

The compounds of formula VII are known from EP 0 289 380 or U.S. Pat. No. 5,017,596 (describing i.a. cizolirtine) as well as EP 1 072 266 or U.S. Pat. No. 6,410,582 included here by reference.

Also provided and currently preferred is a combination according to the application including a compound of general formula (VII), in which R₁₁ is selected from a hydrogen atom or from the group comprised by methyl, ethyl, propyl, isopropyl, butyl, isobutyl, sec-butyl and tert-butyl.

Further provided and currently preferred is a combination according to the application including a compound of general formula (VII), in which R₁₂ is selected from among a group comprised of dimethylaminoethyl, dimethylaminopropyl, diethylaminoethyl, piperidinylethyl, morpholinylpropyl and pirrolidinylethyl.

Still further provided and currently preferred is a combination according to the application including a compound of general formula (VII), in which the compound of general formula (VII) is selected from among a group comprised by:
(±)-5-{α-[2-(dimethylamino)ethoxy]benzyl}-1-methyl-1H-pirazole;
(±)-5-{α-[2-(dimethylamino)ethoxy]benzyl}-1-methyl-1H-pirazole citrate;
(+)-5-{α-[2-(dimethylamino)ethoxy]benzyl}-1-methyl-1H-pirazole;
(−)-5-{α-[2-(dimethylamino)ethoxy]benzyl}-1-methyl-1H-pirazole;
(+)-5-{α-[2-(dimethylamino)ethoxy]benzyl}-1-methyl-1H-pirazole citrate;
(−)-5-{α-[2-(dimethylamino)ethoxy]benzyl}-1-methyl-1H-pirazole citrate;
(±)-5-{α-[2-(dimethylamino)ethoxy]-2-thienylmethyl}-1-methyl-1H-pirazole;
(±)-5-{α-[2-(dimethylamino)ethoxy]-2-thienylmethyl}-1-methyl-1H-pirazole citrate;
(+)-5-{α-[2-(dimethylamino)ethoxy]-2-thienylmethyl}-1-methyl-1H-pirazole;
(−)-5-{α-[2-(dimethylamino)ethoxy]-2-thienylmethyl}-1-methyl-1H-pirazole;
(+)-5-{α-[2-(dimethylamino)ethoxy]-2-thienylmethyl}-1-methyl-1H-pirazole citrate;
(−)-5-{α-[2-(dimethylamino)ethoxy]-2-thienylmethyl}-1-methyl-1H-pirazole citrate.

This application also discloses a process for the production of an aryl or heteroaryl azolylcarbinole derivative according to formula (I) according to the application in which a compound according to formula (VI) 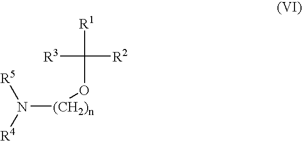
in which
R¹ represents a hydrogen atom or a lower alkyl group from C₁ to C₄;
R represents a phenyl radical or a thienyl radical, with no substitutions or optionally with 1, 2 or 3 equal or different substituents, selected from the group comprised of hydroxyl, fluoride, chloride, bromide, methyl, trifluoromethyl and methoxy or —O-(glucoronic acid);
R³ represents a five-armed nitrogenated aromatic heterocycle that contains one to three nitrogen atoms, without substitutions or optionally substituted by 1 or 2 equal or different substituents selected from a group comprised by fluoride, chloride, bromide and methyl; R⁴ represents H or C₁₋₄-Alkyl; R⁵ represents H or C₁₋₄-Alkyl; n represents 1 to 4 is injected into a mammal (excluding humans), blood of the mammal is collected and the products chromatographically separated and isolated.

As a general remark, the principles of N-glucoronidation are described i.a. in Hawes, (1998); “N+-Glucoronidation A common Pathway in Human Metabolism of drugs with a tertiary amine group”; Drug Metabolism and Disposition; Vol. 26 (9), 830-837; Chiu and Huskey; (1998), “Species Differences in N-Glucoronidation”; Drug Metabolism and Disposition; Vol. 26 (9), 838-847; Kassahun et al.; (1998); “Olanzapine 10-N-Glucoronide”, Drug Metabolism and Disposition; Vol. 26 (9), 848-855; Zenser et al., (1998); “N-Glucoronidation of Benzidine and its metabolites”; Drug Metabolism and Disposition; Vol. 26 (9), 856-859; and Luo et al., (1995) “N+-Glucoronidation of aliphatic tertiary amines in human: antidepressant versus antipsychotic drugs”, Xenobiotica, Vol. 25 (3), 291-301.

The compounds are of high interest as pharmaceutical compounds. The derivatives according to the application are nontoxic and are surprisingly effective in the treatment of depression and in other indications.

Therefore, a further object of the application is a pharmaceutical composition comprising at least one derivative or combination according to the application or one compound selected from
2-((1-methyl-1H-pyrazol-5-yl)(phenyl)methoxy)ethanamine,
2-((1-methyl-1H-pyrazol-5-yl)(phenyl)methoxy)-N-methylethanamine,
N-oxo-2-((1-methyl-1H-pyrazol-5-yl)(phenyl)methoxy)-N,N-dimethylethanamine;
2-((1-methyl-1H-pyrazol-5-yl)(phenyl)methoxy)acetic acid;
2-((1-methyl-1H-pyrazol-5-yl)(thiophen-2-yl)methoxy)-N-methylethanamine;
   optionally in the form of its racemate, pure stereoisomers, especially enantiomers or diastereomers or in the form of mixtures of stereoisomers, especially enantiomers or diastereomers, in any suitable ratio; also optionally in form of a salt, especially a physiologically acceptable salt, or in form of a solvate, especially a hydrate;
   as active ingredient as well as optionally at least one auxiliary material and/or additive.

The compounds 2-((1-methyl-1H-pyrazol-5-yl)(phenyl)methoxy)ethanamine, 2-((1-methyl-1H-pyrazol-5-yl)(phenyl)methoxy)-N-methylethanamine, N-oxo-2-((1-methyl -1H-pyrazol-5-yl)(phenyl)methoxy)-N,N-dimethylethanamine; 2-((1-methyl-1H-pyrazol-5-yl)(phenyl)methoxy) acetic acid; 2-((1-methyl-1H-pyrazol -5-yl)(thiophen-2-yl)methoxy)-N-methylethanamine disclaimed as compounds nevertheless are highly preferred as active ingredients in a pharmaceutical composition being very active. These compounds were described only as metabolites without any pharmacological activity assigned or proven in L. Martinez et al., “Absorption, distribution, metabolism and excretion of cizolirtine, a new analgesic compound, in rat and dog,” Xenobiotica, 1999 (29) 8, 859-871 or in Puig S., et al. J. Pharm. Biomed. Anal. “Validation of a chromatographic method to determine E-6006 and its metabolite E-6332 in rat and dog plasma by solid-phase extraction and capillary gas chromatography.” Application in pharmacokinetics, 24 (2001) 887-896. Surprisingly they showed a very high activity in pharmacological tests.

The auxiliary material and/or additive can be selected from carrier, excipient, support materials, glidants, fillers, solvents, diluents, colorants, taste conditioners like sugars, antioxidants and/or binders. In the case of a suppository this might involve waxes or fatty acid esters or conserving agents, emulsifiers and/or carriers for parenteral application. The selection of these auxiliary materials and/or additives and of the amounts to be used depends upon how the pharmaceutical composition is to be applied.

Examples include here oral or parenteral like pulmonal, nasal, rectal and/or intravenous application. Therefore the pharmaceutical composition according to the application can be adapted for topical or systemical application, especially dermal, subcutaneous, intramuscular, intra-articular and/or intraperitoneal, pulmonal, buccal, sublingual, nasal, percutaneous, vaginal, oral or parenteral, pulmonal, nasal, rectal and/or intravenous application.

For treatment the pharmaceutical composition according to the application might preferably be in the form of a plaster and/or gauze providing an occlusion of the burned or wounded skin.

For oral application preparations in the form of tablets, chewable tablets, dragees, capsules, granules, drops, juices and syrups are suitable. Solutions, suspensions, readily reconstitutable dry preparations and sprays are suitable i.a. for parenteral application. The compounds according to the application as a deposit in a dissolved form or in a patch, optionally with the addition of agents which promote dermal penetration, are examples of suitable percutaneous forms of application. Dermal applications include i.a. an ointment, a gel, a cream, a lotion, a suspension, an emulsion whereas the preferred form for rectal application is a suppository. Therefore, in a preferred object of the application the pharmaceutical composition according to the application is in the form of an ointment, a gel, a cream, a lotion, a suspension, an emulsion, a suppository, a solution, a tablet, a chewable tablet, a dragee, a capsule, a granules, drops, a juice and/or a syrup.

The compounds according to the application can be released in a delayed manner from forms of preparations which can be applied as mentioned above, especially orally, rectally or percutaneously. Retard formulations are preferred objects of the application.

The amount of active ingredient to be administered to the patient varies depending on the weight of the patient, on the type of application, on the indication and on the severity of the illness. 1 to 500 mg of the active ingredient are usually applied per kg.

A further object of the application are pharmaceutical compositions containing at least 0.05 to 90.0 % of active ingredient.

Another important aspect of the application is the use of an aryl or heteroaryl azolylcarbinole derivative or combination according to the application or one compound selected from
2-((1-methyl-1H-pyrazol-5-yl)(phenyl)methoxy)ethanamine,
2-((1-methyl-1H-pyrazol-5-yl)(phenyl)methoxy)-N-methylethanamine,
N-oxo-2-( (1-methyl-1H-pyrazol-5-yl) (phenyl)methoxy)-N,N-dimethylethanamine;
2-((1-methyl-1H-pyrazol-5-yl)(phenyl)methoxy)acetic acid;
2-((1-methyl-1H-pyrazol-5-yl)(thiophen-2-yl)methoxy)-N-methylethanamine;
   optionally in the form of its racemate, pure stereoisomers, especially enantiomers or diastereomers or in the form of mixtures of stereoisomers, especially enantiomers or diastereomers, in any suitable ratio; also optionally in form of a salt, especially a physiologically acceptable salt, or in form of a solvate, especially a hydrate;
   in the preparation of a medicament for the treatment of disorders mediated by excess of substance P; especially anxiety, depression, schizophrenia, manic depressive psychosis, sexual dysfunction, drug addiction, cognitive disorders, locomotive disorders.

Surprisingly it was also found that the compounds according to the application are also active in urinary incontinence. Therefore, a very important aspect of the application is the use of an aryl or heteroaryl azolylcarbinole derivative or combination according to the application or one compound selected from
2-((1-methyl-1H-pyrazol-5-yl)(phenyl)methoxy)ethanamine,
2-( (1-methyl-1H-pyrazol-5-yl) (phenyl)methoxy)-N-methylethanamine,
N-oxo-2-((1-methyl-1H-pyrazol-5-yl)(phenyl)methoxy)-N,N-dimethylethanamine;
2-((1-methyl-1H-pyrazol-5-yl)(phenyl)methoxy)acetic acid;
2-((1-methyl-1H-pyrazol-5-yl)(thiophen-2-yl)methoxy)-N-methylethanamine;
   optionally in the form of its racemate, pure stereoisomers, especially enantiomers or diastereomers or in the form of mixtures of stereoisomers, especially enantiomers or diastereomers, in any suitable ratio; also optionally in form of a salt, especially a physiologically acceptable salt, or in form of a solvate, especially a hydrate; in the preparation of a medicament for the treatment of of Urinary Incontinence: Urge Incontinence, Hyperreflexia; Urinary Stress Incontinence, Mixed Incontinence and Enuresis.

Urinary incontinence, a urinary disorder, is defined as the involuntarily discharge of urine, which can be demonstrated objectively. This functional disorder of bladder is a health problem of increasing social and hygienic relevance for the population that suffers from it. According to our data, urinary incontinence occurs in approximately 1.5 to 5% of men and 10 to 30% of women in the population between 15 and 64 years old. However, if we select the non-hospitalised population sector over 60 years old, the prevalence ranges from 15% to 35% of this population. On the other hand, when hospitalised patients over 60 years old are studied, the incidence is higher. Urinary incontinence affects approximately 2 million of the Spanish population.

Surprisingly it was found that the compounds are also active in pain. Therefore, a further important aspect of the application is the use of an aryl or heteroaryl azolylcarbinole derivative or combination according to the application or one compound selected from
2-((1-methyl-1H-pyrazol-5-yl)(phenyl)methoxy)ethanamine,
2-((1-methyl-1H-pyrazol-5-yl)(phenyl)methoxy)-N-methylethanamine,
N-oxo-2-((1-methyl-1H-pyrazol-5-yl)(phenyl)methoxy)-N,N-dimethylethanamine;
2-((1-methyl-1H-pyrazol-5-yl)(phenyl)methoxy)acetic acid;
2-((1-methyl-1H-pyrazol-5-yl)(thiophen-2-yl)methoxy)-N-methylethanamine;
   optionally in the form of its racemate, pure stereoisomers, especially enantiomers or diastereomers or in the form of mixtures of stereoisomers, especially enantiomers or diastereomers, in any suitable ratio; also optionally in form of a salt, especially a physiologically acceptable salt, or in form of a solvate, especially a hydrate; in the preparation of a medicament for the treatment of pain, including acute pain, chronic pain, neuropathic pain and visceral pain especially pain of moderate to high intensity; sciatica, lumbago, dorsalgia, sprains, fractures, dislocations, postoperative pain, and pain of dental origin.

This application also discloses the use of an aryl or heteroaryl azolylcarbinole derivative or combination according to the application or one compound selected from
2-((1-methyl-1H-pyrazol-5-yl)(phenyl)methoxy)ethanamine,
2-((1-methyl-1H-pyrazol-5-yl)(phenyl)methoxy)-N-methylethanamine,
N-oxo-2-((1-methyl-1H-pyrazol-5-yl)(phenyl)methoxy)-N,N-dimethylethanamine;
2-((1-methyl-1H-pyrazol-5-yl)(phenyl)methoxy)acetic acid;
2-((1-methyl-1H-pyrazol-5-yl)(thiophen-2-yl)methoxy)-N-methylethanamine;
   optionally in the form of its racemate, pure stereoisomers, especially enantiomers or diastereomers or in the form of mixtures of stereoisomers, especially enantiomers or diastereomers, in any suitable ratio; also optionally in form of a salt, especially a physiologically acceptable salt, or in form of a solvate, especially a hydrate;
   in the preparation of a medicament for the treatment of neuropathic inflammation: diabetes, asthma, cystitis, gingivitis, migraine, dermatitis, rhinitis, psoriasis, inflammation of sciatic and lumbar nerves, gastrointestinal processes, ocular inflammation; the treatment of relative respiratory diseases,: cough, bronchitis, chronic obstructive pulmonary diseases, allergic rhinitis, asthma.

For a further clarification the compounds according to the application together with the disclaimed compounds are highly interesting active compounds in a pharmaceutical composition, but are not yet described as such.

Also provided is therefore a pharmaceutical composition comprising as active ingredient at least one aryl or heteroaryl azolylcarbinole derivative of general formula (I), (II), (III) or (IV) 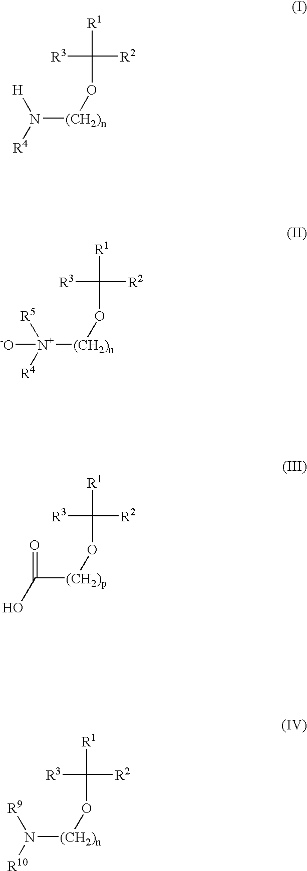
in which
R¹ represents a hydrogen atom or a lower alkyl group from C₁ to C₄; R² represents a phenyl radical or a thienyl radical, with no substitutions or optionally with 1, 2 or 3 equal or different substituents, selected from the group comprised of hydroxy, fluoride, chloride, bromide, methyl, trifluoromethyl and methoxy, or —O-(glucoronic acid); R³ represents a five-armed nitrogenated aromatic heterocycle that contains one to three nitrogen atoms, without substitutions or optionally substituted by 1 or 2 equal or different substituents selected from a group comprised by fluoride, chloride, bromide and methyl; R⁴ represents H or C₁₋₄-Alkyl; R⁵ represents H or C₁₋₄-Alkyl; R⁹ is glucoronic acid; R¹⁰ represents C₁₋₄-Alkyl; and n is 1 to 4 or p is 1 to 3
   optionally in the form of its racemate, pure stereoisomers, especially enantiomers or diastereomers or in the form of mixtures of stereoisomers, especially enantiomers or diastereomers, in any suitable ratio; in the form shown or in form of the acid or base or in form of a salt, especially a physiologically acceptable salt, or in form of a solvate, especially a hydrate; as well as additional pharmaceutical ingredients.

Preferably, a pharmaceutical composition comprising an aryl or heteroaryl azolylcarbinole derivative of general formula (I), (II), (III) or (IV) in which R₁ is selected from hydrogen or from a group consisting of methyl, ethyl, propyl, isopropyl, butyl, isobutyl, sec-butyl and tert-butyl.

Preferably, a pharmaceutical composition comprising an aryl or heteroaryl azolylcarbinole derivative of general formula (I), (II), (III) or (IV) in which R₄ is selected from hydrogen, methyl or ethyl, especially methyl or hydrogen.

Preferably, a pharmaceutical composition comprising an aryl or heteroaryl azolylcarbinole derivative of general formula (II) in which R₅ is selected from hydrogen, methyl or ethyl, especially methyl or hydrogen.

Preferably, a pharmaceutical composition comprising an aryl or heteroaryl azolylcarbinole derivative of general formula (I), (II), (III) or (IV), in which R³ is selected from 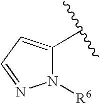
with R⁶ being selected from hydrogen, fluoride, chloride, bromide and methyl. There it is preferred if R⁶ is hydrogen or methyl.

Preferably, a pharmaceutical composition comprising an aryl or heteroaryl azolylcarbinole derivative of general formula (I), (II), (III) or (IV) in which R² is selected from: 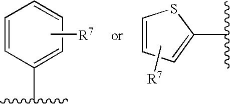
with R⁷ being selected from the group consisting of hydroxyl, hydrogen, fluoride, chloride, bromide, methyl, trifluoromethyl and methoxy, or —O-(glucoronic acid).

There it is preferred that R⁷ is hydrogen or hydroxyl, or —O-(glucoronic acid).

Highly preferably, is a pharmaceutical composition comprising an aryl or heteroaryl azolylcarbinole derivative of general formula (I) or (II) in which the compound is a compound according to one of the general formulas (Ia) or (IIa) 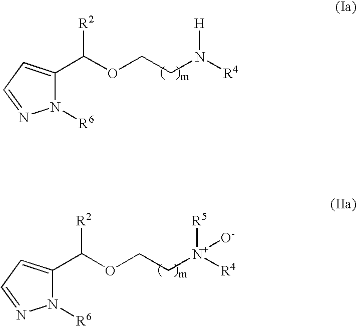
in which
m is 1 or 2; R² represents a phenyl radical or a thienyl radical, with no substitutions or optionally with 1, 2 or 3 equal or different substituents, selected from the group comprised of hydroxyl, fluoride, chloride, bromide, methyl, trifluoromethyl and methoxy; R⁴ represents H or C₁₋₄-Alkyl; R⁵ represents H or C₁₋₄-Alkyl; and R⁶ is selected from hydrogen, fluoride, chloride, bromide and methyl.

Preferably, a pharmaceutical composition comprising an aryl or heteroaryl azolylcarbinole derivative of general formula (Ia) or (IIa) in which
R² is selected from: 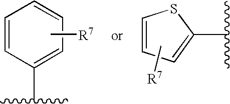
   with R₇ being selected from the group consisting of hydroxyl, hydrogen, fluoride, chloride, bromide, methyl, trifluoromethyl and methoxy.

There it is preferred that R⁷ is hydrogen or hydroxyl.

Preferred is a pharmaceutical composition comprising an aryl or heteroaryl azolylcarbinole derivative of general formula (Ia) or (IIa) in which R⁴ is methyl or ethyl, preferably methyl.

Preferred is a pharmaceutical composition comprising an aryl or heteroaryl azolylcarbinole derivative of general formula (IIa), in which R⁵ is hydrogen, methyl or ethyl, preferably hydrogen or methyl.

Preferred is a pharmaceutical composition comprising an aryl or heteroaryl azolylcarbinole derivative of general formula (Ia) or (IIa) in which R⁶ is hydrogen or methyl.

Highly prererred is a pharmaceutical composition comprising an aryl or heteroaryl azolylcarbinole derivative of general formula (I) or (II) in which the compound is a compound according to one of the general formulas (Ib), (Ic), (IIb) or (IIc) 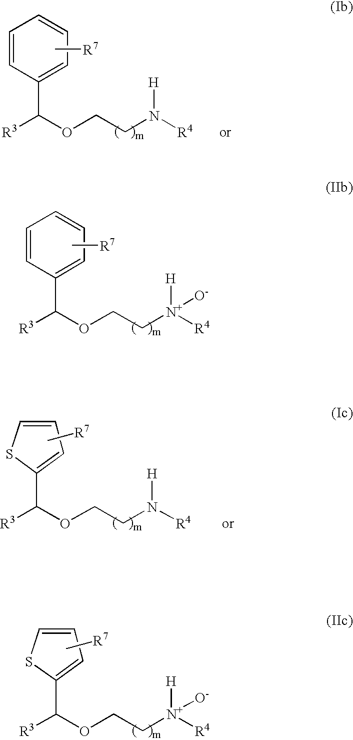
in which
m is 1 or 2;
R³ represents a five-armed nitrogenated aromatic heterocycle that contains one to three nitrogen atoms, without substitutions or optionally substituted by 1 or 2 equal or different substituents selected from a group comprised by fluoride, chloride, bromide and methyl;
R⁴ represents H or C₁₋₄-Alkyl;
R⁵ represents H or C₁₋₄-Alkyl; and
R⁷ is selected from the group consisting of hydroxyl, hydrogen, fluoride, chloride, bromide, methyl, trifluoromethyl and methoxy.

Preferred is a pharmaceutical composition comprising an aryl or heteroaryl azolylcarbinole derivative of general formula (Ib), (Ic), (IIb) or (IIc), in which R⁷ is hydrogen or hydroxyl.

Preferred is a pharmaceutical composition comprising an aryl or heteroaryl azolylcarbinole derivative of general formula (Ib), (Ic), (IIb) or (IIc), in which R³ is selected from 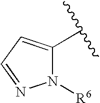
with R⁶ being selected from hydrogen, fluoride, chloride, bromide and methyl.

There it is preferred if R⁶ is hydrogen or methyl.

Preferred is a pharmaceutical composition comprising an aryl or heteroaryl azolylcarbinole derivative of general formula (Ib), (Ic), (IIb) or (IIc), in which R⁴ is methyl or ethyl, preferably methyl.

Preferred is a pharmaceutical composition comprising an aryl or heteroaryl azolylcarbinole derivative of general formula (IIb) or (IIc), in which R⁵ is hydrogen, methyl or ethyl, preferably hydrogen or methyl.

Highly preferred is a pharmaceutical composition comprising an aryl or heteroaryl azolylcarbinole derivative of general formula (I) or (II) in which the compound is a compound according to one of the general formulas (Id), (Ie), (IId) or (IIe) 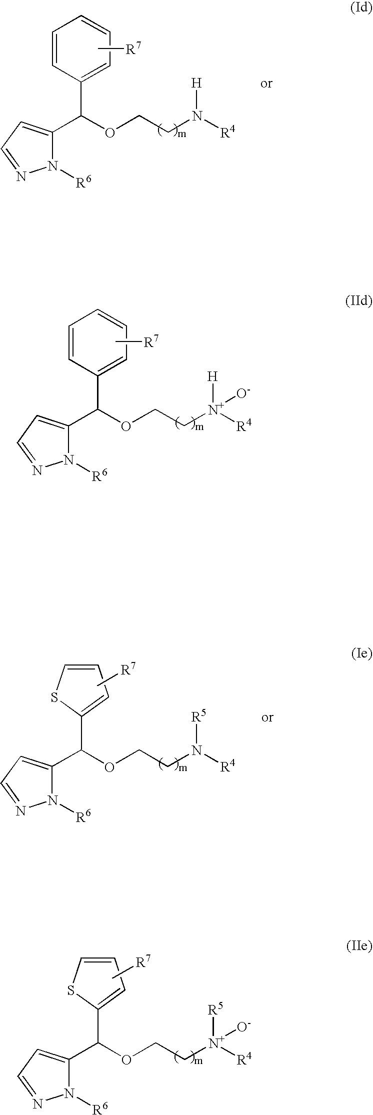
in which
m is 1 or 2;
R⁴ is selected from H or lower C₍₁₄₎-Alkyl;
R⁵ is selected from H or lower C₍₁₋₄₎-Alkyl;
R⁶ is selected from hydrogen, fluoride, chloride, bromide and methyl; and
R₇ is selected from the group consisting of hydroxyl, hydrogen, fluoride, chloride, bromide, methyl, trifluoromethyl and methoxy.

Preferred is a pharmaceutical composition comprising an aryl or heteroaryl azolylcarbinole derivative of general formula (Id), (Ie), (IId) or (IIe), in which R⁷ is hydrogen or hydroxyl.

Preferred is a pharmaceutical composition comprising an aryl or heteroaryl azolylcarbinole derivative of general formula (Id), (Ie), (IId) or (IIe), in which R⁶ is hydrogen or methyl.

Preferred is a pharmaceutical composition comprising an aryl or heteroaryl azolylcarbinole derivative of general formula (Id), (Ie), (IId) or (IIe), in which R⁴ is methyl or ethyl, preferably methyl.

Preferably, a pharmaceutical composition comprising the salts, the physiologically acceptable salts of the new compounds. In this regards it is highly preferred that the compounds according to general formula (II) are in a form according to general formula (V) 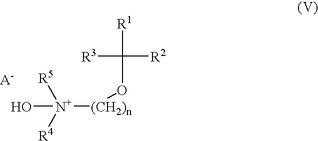
in which
R¹ represents a hydrogen atom or a lower alkyl group from C₁to C₄;
R represents a phenyl radical or a thienyl radical, with no substitutions or optionally with 1, 2 or 3 equal or different substituents, selected from the group comprised of hydroxyl, fluoride, chloride, bromide, methyl, trifluoromethyl and methoxy;
R³ represents a five-armed nitrogenated aromatic heterocycle that contains one to three nitrogen atoms, without substitutions or optionally substituted by 1 or 2 equal or different substituents selected from a group comprised by fluoride, chloride, bromide and methyl;
R⁴ represents H or C₁₋₄-Alkyl;
R⁵ represents H or C₁₋₄-Alkyl.

Highly preferably, the new aryl or heteroaryl azolylcarbinole derivatives comprised in the pharmaceutical composition according to this application are selected from:
2-((1-methyl-1H-pyrazol-5-yl)(phenyl)methoxy)-N-methylethanamine;
(±)-2-((1-methyl-1H-pyrazol-5-yl)(phenyl)methoxy)-N-methylethanamine;
(+)-2-((1-methyl-1H-pyrazol-5-yl)(phenyl)methoxy)-N-methylethanamine;
(−)-2-((1-methyl-1H-pyrazol-5-yl)(phenyl)methoxy)-N-methylethanamine;
2-((1-methyl-1H-pyrazol-5-yl)(phenyl)methoxy)ethanamine;
(±)-2-((1-methyl-1H-pyrazol-5-yl)(phenyl)methoxy)ethanamine;
(+)-2-((1-methyl-1H-pyrazol-5-yl)(phenyl)methoxy)ethanamine;
(−)-2-((1-methyl-1H-pyrazol-5-yl)(phenyl)methoxy)ethanamine;
4-((2-(dimethylamino)ethoxy)(1-methyl-1H-pyrazol-5-yl)methyl)phenol;
(±)-4-((2-(dimethylamino)ethoxy)(1-methyl-1H-pyrazol-5-yl)methyl)phenol;
(+)-4-((2-(dimethylamino)ethoxy)(1-methyl-1H-pyrazol-5-yl)methyl)phenol;
(−)-4-((2-(dimethylamino)ethoxy)(1-methyl-1H-pyrazol-5-yl)methyl)phenol;
N,N-dimethyl-2-(phenyl(1H-pyrazol-5-yl)methoxy)ethanamine;
(±)-N,N-dimethyl-2-(phenyl(1H-pyrazol-5-yl)methoxy)ethanamine;
(+)-N,N-dimethyl-2-(phenyl(1H-pyrazol-5-yl)methoxy)ethanamine;
(−)-N,N-dimethyl-2-(phenyl(1H-pyrazol-5-yl)methoxy)ethanamine;
2-((1-methyl-1H-pyrazol-5-yl)(phenyl)methoxy)acetic acid;
(±)-2-((1-methyl-1H-pyrazol-5-yl)(phenyl)methoxy)acetic acid;
(+)-2-((1-methyl-1H-pyrazol-5-yl)(phenyl)methoxy)acetic acid;
(−)-2-((1-methyl-1H-pyrazol-5-yl)(phenyl)methoxy)acetic acid;
6-(4-((2-(dimethylamino)ethoxy)(1-methyl-1H-pyrazol-5-yl)methyl)phenoxy)-tetrahydro -3,4,5-trihydroxy-2H-pyran-2-carboxylic acid
(±)-6-(4-((2-(dimethylamino)ethoxy)(1-methyl-1H-pyrazol -5-yl)methyl)phenoxy)-tetrahydro-3,4,5-trihydroxy -2H-pyran-2-carboxylic acid
(+)-6-(4-((2-(dimethylamino)ethoxy)(1-methyl-1H-pyrazol -5-yl)methyl)phenoxy)-tetrahydro-3,4,5-trihydroxy -2H-pyran-2-carboxylic acid
(−)-6-(4-((2-(dimethylamino)ethoxy)(1-methyl-1H-pyrazol -5-yl)methyl)phenoxy)-tetrahydro-3,4,5-trihydroxy -2H-pyran-2-carboxylic acid
6-(4-((2-(methylamino)ethoxy)(1-methyl-1H-pyrazol-5-yl)methyl)phenoxy)-tetrahydro -3,4,5-trihydroxy-2H-pyran -2-carboxylic acid
(±)-(6-(4-((2-(methylamino)ethoxy)(1-methyl-1H-pyrazol -5-yl)methyl)phenoxy)-tetrahydro-3,4,5-trihydroxy-2H-pyran -2-carboxylic acid
(+)-6-(4-((2-(methylamino)ethoxy)(1-methyl-1H-pyrazol -5-yl)methyl)phenoxy)-tetrahydro-3,4,5-trihydroxy-2H-pyran -2-carboxylic acid
(−)-6-(4-((2-(methylamino)ethoxy)(1-methyl-1H-pyrazol -5-yl)methyl)phenoxy)-tetrahydro-3,4,5-trihydroxy-2H-pyran -2-carboxylic acid
6-(N-(2-((1-methyl-1H-pyrazol-5-yl)(phenyl)methoxy)ethyl)-N-methylamino)-tetrahydro -3,4,5-trihydroxy-2H-pyran-2-carboxylic acid
(±)-6-(N-(2-((1-methyl-1H-pyrazol-5-yl)(phenyl)methoxy)ethyl)-N-methylamino)-tetrahydro -3,4,5-trihydroxy-2H-pyran-2-carboxylic acid
(+)-6-(N-(2-((1-methyl-1H-pyrazol-5-yl)(phenyl)methoxy)ethyl)-N-methylamino)-tetrahydro -3,4,5-trihydroxy-2H-pyran-2-carboxylic acid
(−)-6-(N-(2-((1-methyl-1H-pyrazol-5-yl)(phenyl)methoxy)ethyl)-N-methylamino)-tetrahydro -3,4,5-trihydroxy-2H-pyran-2-carboxylic acid
2-((1-methyl-1H-pyrazol-5-yl)(thiophen-2-yl)methoxy)-N-methylethanamine;
(±)-2-((1-methyl-1H-pyrazol-5-yl)(thiophen-2-yl)methoxy)-N-methylethanamine;
(+)-2-((1-methyl-1H-pyrazol-5-yl)(thiophen-2-yl)methoxy)-N-methylethanamine;
(−)-2-((1-methyl-1H-pyrazol-5-yl)(thiophen-2-yl)methoxy)-N-methylethanamine;
2-((1-methyl-1H-pyrazol-5-yl)(thiophen-2-yl)methoxy)ethanamine;
(±)-2-((1-methyl-1H-pyrazol-5-yl)(thiophen-2-yl)methoxy)ethanamine;
(+)-2-((1-methyl-1H-pyrazol-5-yl)(thiophen-2-yl)methoxy)ethanamine;
(−)-2-((1-methyl-1H-pyrazol-5-yl)(thiophen-2-yl)methoxy)ethanamine;
2-((1H-pyrazol-5-yl)(thiophen-2-yl)methoxy)-N,N-dimethylethanamine;
(±)-2-((1H-pyrazol-5-yl)(thiophen-2-yl)methoxy)-N,N-dimethylethanamine;
(+)-2-((1H-pyrazol-5-yl)(thiophen-2-yl)methoxy)-N,N-dimethylethanamine;
(−)-2-((1H-pyrazol-5-yl)(thiophen-2-yl)methoxy)-N,N-dimethylethanamine;
5-((2-(dimethylamino)ethoxy)(1-methyl-1H-pyrazol-5-yl)methyl)thiophen -3-ol;
(±)-5-((2-(dimethylamino)ethoxy)(1-methyl-1H-pyrazol-5-yl)methyl)thiophen -3-ol;
(+)-5-((2-(dimethylamino)ethoxy)(1-methyl-1H-pyrazol-5-yl)methyl)thiophen -3-ol;
(−)-5-((2-(dimethylamino)ethoxy)(1-methyl-1H-pyrazol-5-yl)methyl)thiophen -3-ol;
5-((2-(dimethylamino)ethoxy)(1-methyl-1H-pyrazol-5-yl)methyl)thiophen -2-ol;
(±)-5-((2-(dimethylamino)ethoxy)(1-methyl-1H-pyrazol-5-yl)methyl)thiophen -2-ol;
(+)-5-((2-(dimethylamino)ethoxy)(1-methyl-1H-pyrazol-5-yl)methyl)thiophen -2-ol;
(−)-5-((2-(dimethylamino)ethoxy)(1-methyl-1H-pyrazol-5-yl)methyl)thiophen -2-ol;
2-((1-methyl-1H-pyrazol-5-yl)(thiophen-2-yl)methoxy)acetic acid;
(±)-2-((1-methyl-1H-pyrazol-5-yl)(thiophen-2-yl)methoxy)acetic acid;
(+)-2-((1-methyl-1H-pyrazol-5-yl)(thiophen-2-yl)methoxy)acetic acid;
(−)-2-((1-methyl-1H-pyrazol-5-yl)(thiophen-2-yl)methoxy)acetic acid;
6-((5-((2-(dimethylamino)ethoxy)(1-methyl-1H-pyrazol-5-yl)methyl)thiophen -3-yl)methyl)-tetrahydro-3,4,5-trihydroxy -2H-pyran-2-carboxylic acid
(±)-6-((5-((2-(dimethylamino)ethoxy)(1-methyl-1H-pyrazol -5-yl)methyl)thiophen-3-yl)methyl)-tetrahydro -3,4,5-trihydroxy-2H-pyran-2-carboxylic acid
(+)-6-((5-((2-(dimethylamino)ethoxy)(1-methyl-1H-pyrazol -5-yl)methyl)thiophen-3-yl)methyl)-tetrahydro -3,4,5-trihydroxy-2H-pyran-2-carboxylic acid
(−)-6-((5-((2-(dimethylamino)ethoxy)(1-methyl-1H-pyrazol -5-yl)methyl)thiophen-3-yl)methyl)-tetrahydro -3,4,5-trihydroxy-2H-pyran-2-carboxylic acid
2-(methylamino)ethoxy)(1-methyl-1H-pyrazol-5-yl)methyl)thiophen -3-yl)methyl)-tetrahydro-3,4,5-trihydroxy -2H-pyran-2-carboxylic acid
(±)-2-(methylamino)ethoxy)(1-methyl-1H-pyrazol-5-yl)methyl)thiophen -3-yl)methyl)-tetrahydro-3,4,5-trihydroxy -2H-pyran-2-carboxylic acid
(+)-2-(methylamino)ethoxy)(1-methyl-1H-pyrazol-5-yl)methyl)thiophen -3-yl)methyl)-tetrahydro-3,4,5-trihydroxy -2H-pyran-2-carboxylic acid
(−)-2-(methylamino)ethoxy)(1-methyl-1H-pyrazol-5-yl)methyl)thiophen -3-yl)methyl)-tetrahydro-3,4,5-trihydroxy -2H-pyran-2-carboxylic acid
6-(N-(2-((1-methyl-1H-pyrazol-5-yl)(thiophen-2-yl)methoxy)ethyl)-N-methylamino)-tetrahydro -3,4,5-trihydroxy -2H-pyran-2-carboxylic acid
(±)-6-(N-(2-((1-methyl-1H-pyrazol-5-yl)(thiophen-2-yl)methoxy)ethyl)-N-methylamino)-tetrahydro -3,4,5-trihydroxy -2H-pyran-2-carboxylic acid
(+)-6-(N-(2-((1-methyl-1H-pyrazol-5-yl)(thiophen-2-yl)methoxy)ethyl)-N-methylamino)-tetrahydro -3,4,5-trihydroxy -2H-pyran-2-carboxylic acid
(−)-6-(N-(2-((1-methyl-1H-pyrazol-5-yl)(thiophen-2-yl)methoxy)ethyl)-N-methylamino)-tetrahydro -3,4,5-trihydroxy -2H-pyran-2-carboxylic acid;
   optionally also in form of a corresponding N-oxide according to any of formulas (II), (IIa), (IIb), (IIc), (IId) or (IIe) and optionally in form of a salt, especially a physiologically acceptable salt, most preferably the citrate or in form of a solvate, especially a hydrate.

Another aspect of the this application is a pharmaceutical composition comprising a combination of an aryl or heteroaryl azolylcarbinole derivative according to the application and a compound of general formula VII 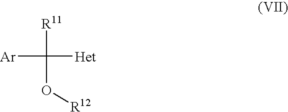
in which
Ar represents a phenyl radical or a thienyl radical, with no substitutions or optionally with 1, 2 or 3 equal or different substituents, selected from the group comprised of fluoride, chloride, bromide, methyl, trifluoromethyl and methoxy;
R₁₁ represents a hydrogen atom or a lower alkyl group from C₁ to C₄;
R₁₂ represents a dialkyl(C₁-C₄)aminoalkyl (C₂-C₃), or azaheterocyclylalkyl (C₂-C₃) radical; and
Het represents a five-armed nitrogenated aromatic heterocycle that contains one to three nitrogen atoms, without substitutions or optionally substituted by 1 or 2 equal or different substituents selected from a group comprised by fluoride, chloride, bromide and methyl.

The compounds of formula VII are known from EP 0 289 380 or U.S. Pat. No. 5,017,596 (describing i.a. cizolirtine) as well as EP 1 072 266 or U.S. Pat. No. 6,410,582 included here by referrence.

Preferred is a pharmaceutical composition comprising a combination according to the application including a compound of general formula (VII), in which R₁₁ is selected from a hydrogen atom or from the group comprised by methyl, ethyl, propyl, isopropyl, butyl, isobutyl, sec-butyl and tert-butyl.

Preferred is a pharmaceutical composition comprising a combination according to the application including a compound of general formula (VII), in which R₁₂ is selected from among a group comprised of dimethylaminoethyl, dimethylaminopropyl, diethylaminoethyl, piperidinylethyl, morpholinylpropyl and pirrolidinylethyl.

Preferred is a pharmaceutical composition comprising a combination according to the application including a compound of general formula (VII), in which the compound of general formula (VII) is selected from among a group comprised by:
(±)-5-{α-[2-(dimethylamino)ethoxy]benzyl}-1-methyl-1H-pirazole;
(±)-5-{α-[2-(dimethylamino)ethoxy]benzyl}-1-methyl-1H-pirazole citrate;
(+)-5-{α-[2-(dimethylamino)ethoxy]benzyl}-1-methyl-1H-pirazole;
(−)-5-{α-[2-(dimethylamino)ethoxy]benzyl}-1-methyl-1H-pirazole;
(+)-5-{α-[2-(dimethylamino)ethoxy]benzyl}-1-methyl-1H-pirazole citrate;
(−)-5-{α-[2-(dimethylamino)ethoxy]benzyl}-1-methyl-1H-pirazole citrate;
(±)-5-{α-[2-(dimethylamino)ethoxy]-2-thienylmethyl}-1-methyl-1H-pirazole;
(±)-5-{α-[2-(dimethylamino)ethoxy]-2-thienylmethyl}-1-methyl-1H-pirazole citrate;
(+)-5-{α-[2-(dimethylamino)ethoxy]-2-thienylmethyl}-1-methyl-1H-pirazole;
(−)-5-{α-[2-(dimethylamino)ethoxy]-2-thienylmethyl}-1-methyl-1H-pirazole;
(+)-5-{α-[2-(dimethylamino)ethoxy]-2-thienylmethyl}-1-methyl-1H-pirazole citrate;
(−)-5-{α-[2-(dimethylamino)ethoxy]-2-thienylmethyl}-1-methyl-1H-pirazole citrate.

These medicaments are also useful in the preparation of a medicament for the treatment of disorders mediated by excess of substance P; especially anxiety, depression, schizophrenia, manic depressive psychosis, sexual dysfunction, drug addiction, cognitive disorders, locomotive disorders.

Or pain, including acute pain, chronic pain, neuropathic pain and visceral pain especially pain of moderate to high intensity; sciatica, lumbago, dorsalgia, sprains, fractures, dislocations, postoperative pain, and pain of dental origin.

Or urinary Incontinence: Urge Incontinence, Hyperreflexia; Urinary Stress Incontinence, Mixed Incontinence and Enuresis.

Or neuropathic inflammation: diabetes, asthma, cystitis, gingivitis, migraine, dermatitis, rhinitis, psoriasis, inflammation of sciatic and lumbar nerves, gastrointestinal processes, ocular inflammation; the treatment of relative respiratory diseases,: cough, bronchitis, chronic obstructive pulmonary diseases, allergic rhinitis, asthma.

This applies also to (+)-N,N-dimethyl-2-(phenyl(1H-pyrazol -5-yl)methoxy)ethanamine or (−)-N,N-dimethyl-2-(phenyl( 1H-pyrazol-5-yl)methoxy)ethanamine, whose use as active ingredients in a pharmaceutical composition is also claimed.

1. A method of treating a subject suffering from a form of urinary incontinence which comprises administering to the subject an amount of a compound having the structure: 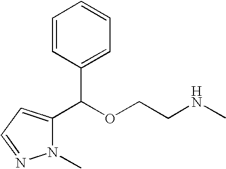
effective to treat the subject, wherein the compound is administered in a suitable form.

2. The method of claim 1, wherein the compound is in the form of a racemic mixture.

3. The method of claim 1, wherein the compound is in the form of a pure stereoisomer or as a mixture of stereoisomers in a suitable relative ratio.

4. The method of claim 1, wherein the compound is in the form of an enantiomer, a diastereomer, or a mixture of enantiomers and/or diasteromers in a suitable relative ratio.

5. The method of claim 1, wherein the compound is in the form of an acid, a base, a physiologically acceptable salt, or a solvate.

6. The method of claim 5, wherein the compound is in the form of a solvate, and the solvate is a hydrate.

7. The method of claim 1, wherein the compound is in the form of an enantiomer selected from the group consisting of:
(R)-(+)-2-((1-methyl-1H-pyrazol-5-yl)(phenyl)methoxy-N-methylethanamine
(S)-(−)-2-((1-methyl-1H-pyrazol-5-yl)(phenyl)methoxy-N-methylethanamine
(R)-(+)-2-((1-methyl-1H-pyrazol-5-yl)(phenyl)methoxy-N-methylethanamine citrate
(S)-(−)-2-((1-methyl-1H-pyrazol-5-yl)(phenyl)methoxy-N-methylethanamine citrate

8. The method of claim 1, wherein the compound is in the form of a pharmaceutically acceptable salt which is a citrate.

9. The method of claim 1, wherein the subject is a human being.

10. The method of claim 1, wherein the effective amount is between 0.167 and 13.333 mg/kg body weight of the subject/day.

11. The method of claim 10, wherein the effective amount is between 0.167 and 3.333 mg/kg body weight of the subject/day.

12. The method of claim 10, wherein the effective amount is between 0.333 and 1.667 mg/kg body weight of the subject/day.

13. The method claim 1, wherein the effective amount is between 10 and 800 mg administered daily.

14. The method of claim 13, wherein the effective amount is between 10 and 200 mg administered daily.

15. The method of claim 13, wherein the effective amount is between 20 and 100 mg administered daily.

16. The method of claim 13, wherein the effective amount of 200 mg administered daily.

17. The method of claim 13, wherein the effective amount is of 100 mg administered daily.

18. The method of claim 13, wherein the effective amount is of 50 mg administered daily.

19. The method of claim 13, wherein the effective amount is of 20 mg administered daily.

20. The method of claim 1, wherein the compound is administered twice per day.

21. The method of claim 1, wherein the compound is present in a formulation that contains a coating agent and the formulation is administered daily.

22. The method of claim 21, wherein the coating agent is a controlled release coating agent.

23. The method of claim 21, wherein the formulation comprises any of the following: sodium croscarmelose; colloidal silica dioxide; a salt with stearic acid; providone; microcrystalline cellulose; lactose monohydrate; or polyethylene glycol.

24. The method of claim 1, wherein the compound being is administered in the form of a tablet or capsule.

25. The method of claim 24, wherein the compound is administered in the form of an immediate release formulation.

26. The method of claime 1, wherein the subject is a woman.

27. The method of claim 26, wherein the woman is an elderly woman.

28. The method of claims 1, wherein the subject is a man.

29. The method of claim 28, wherein the man is an elderly man.

30. The method of claim 1, wherein the subject is a child.

31. The method of claim 1, wherein the form of urinary incontinence is urge urinary incontinence.

32. The method of claim 1, wherein the form of urinary incontinence is stress urinary incontinence or urinary stress incontinence.

33. The method of claim 1, wherein the form of urinary incontinence is hyperreflexive urinary incontinence.

34. The method of claim 1, wherein the form of urinary incontinence is enuresis.
